(19) United States
(12) Patent Application Publication
(10) Pub. No.: US 2025/0118426 A1
(43) Pub. Date: Apr. 10, 2025

(54) TECHNIQUES FOR IMPROVING EFFICIENCY OF DETECTION, COMMUNICATION, AND SECONDARY EVALUATION OF HEALTH EVENTS

(71) Applicant: Medtronic, Inc., Minneapolis, MN (US)

(72) Inventors: Jeffrey M. Gillberg, Minneapolis, MN (US); Kevin T. Ousdigian, Shoreview, MN (US); Shantanu Sarkar, Roseville, MN (US); Sean R. Landman, Minneapolis, MN (US); Abhijit Kadrolkar, St. Paul, MN (US); Christopher T. House, Pine Island, MN (US); Traci K. Washburn, Lino Lakes, MN (US); David I. Siegfried, Edina, MN (US); Abhijit P. Jejurkar, Plymouth, MN (US); Paul G. Krause, Mahtomedi, MN (US)

(21) Appl. No.: 18/832,365

(22) PCT Filed: Feb. 7, 2023
(86) PCT No.: PCT/US2023/012481

§ 371 Date: Jul. 23, 2024

Related U.S. Application Data
(60) Provisional application No. 63/267,824, filed on Feb. 10, 2022.

(51) Int. Cl. G16H 40/67; A61B 5/00; A61B 5/363

(52) U.S. Cl. CPC G16H 40/67; A61B 5/0006; A61B 5/0031; A61B 5/363; A61B 5/6861

(57) ABSTRACT

Techniques are described for initiating a change to rules used by a medical device to identify a plurality of episodes based on a determination that an amount of the plurality of episodes classified as a classification for which transmission of the episode data from the medical device to the computing device was unnecessary satisfies at least one criterion.

This application claims the benefit of priority from U.S. Provisional Application Ser. No. 63/267,824, filed Feb. 10, 2022, the entire content of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

In some cases, such devices are configured to detect acute health events based on the physiological signals, such as episodes of cardiac arrhythmia, myocardial infarction, stroke, or seizure. Example arrhythmia types include cardiac arrest (e.g., asystole), ventricular tachycardia (VT), and ventricular fibrillation (VF). The devices may store ECG and other physiological signal data collected during a time period including an episode as episode data. Such acute health events are associated with significant rates of death, particularly if not treated quickly.

For example, VF and other malignant tachyarrhythmias are the most commonly identified arrhythmia in sudden cardiac arrest (SCA) patients. If this arrhythmia continues for more than a few seconds, it may result in cardiogenic shock and cessation of effective blood circulation. The survival rate from SCA decreases between 7 and 10 percent for every minute that the patient waits for defibrillation. Consequently, sudden cardiac death (SCD) may result in a matter of minutes.

### SUMMARY

In general, the disclosure describes techniques for detection of acute health events, such as sudden cardiac arrest (SCA), by monitoring patient parameter data. More particularly, the disclosure describes techniques for applying rules, which may include one or more machine learning models, to patient parameter data to detect acute health events. The techniques include configuring rules and/or the application of the rules to the patient parameter data in order to improve the efficiency and effectiveness of the detection of acute health events.

Unlike conventional acute health event (e.g., SCA) detection systems, the techniques and systems of this disclosure may use a machine learning model to more accurately determine whether the acute health event is present in ECG and/or other patient parameter data collected by a medical device. In some examples, the machine learning model is trained with a set of training instances, where one or more of the training instances comprise data that indicate relationships between patient parameter and classifications related to the acute health event, e.g., related to potentially lethal cardiac arrhythmias. Because the machine learning model is trained with potentially thousands or millions of training instances, the machine learning model may, for example, reduce the amount of classification error in classifying ECG data as different arrhythmia classifications when compared to conventional detection systems.

Additionally, the techniques and systems of this disclosure may be implemented with an implantable medical device (IMD) that can continuously (e.g., on a periodic or triggered basis without human intervention) sense the ECG and/or other patient parameter data while subcutaneously implanted in a patient over months or years and perform numerous operations per second on patient parameter data to enable the systems herein to detect acute health events. Using techniques of this disclosure with an IMD may be advantageous when a physician cannot be continuously present with the patient over weeks or months to evaluate the patient parameter data and/or where performing the operations on the ECG and/or other patient parameter described herein (e.g., application of a machine learning model) on weeks or months of data could not practically be performed in the mind of a physician.

In some examples, processing circuitry of a computing device configured to wirelessly communicate with an IMD or other medical device applies a machine learning model to patient parameter data as a second set of rules to confirm or reject detection of an acute health event by the medical device using a first set of rules. Reducing classification errors for acute health events with a machine learning model implementing techniques of this disclosure may provide one or more technical and clinical advantages. In some examples, the sensitivity/specificity balance of the first set of rules used by the medical device to initially detect acute health events may be dynamically adjusted to address changing patient conditions, e.g., increased risk of the acute health event or increased presence of false positives, which may result in an appropriate amount of review of the patient parameter data by a clinician for the patient's current condition. Improved specificity and sensitivity may increase the ability of another device, user, and/or clinician to rely on the accuracy of the system's assessment of the patient's condition and improve resulting treatment of the patient and patient outcomes.

In some examples, a computing device comprises communication circuitry, processing circuitry, and memory. The memory comprises memory comprising program instructions that, when executed by the processing circuitry, cause the processing circuitry to; apply a second set of rules to episode data wirelessly received from a medical device for each of a plurality of episodes via the communication circuitry, the episode data stored by the medical device for the episodes in response to detecting a health event based on application of a first set of rules by the medical device to sensed data sensed by the medical device; classify each episode of the plurality of episodes as one of a plurality of classifications based on the application of the second set of rules to the episode data, wherein at least one of the plurality of classifications comprises a classification for which transmission of the episode data from the medical device to the computing device was unnecessary; determine that an amount of the plurality of episodes classified as the classification for which transmission of the episode data from the medical device to the computing device was unnecessary satisfies at least one criterion; and initiate a change to the first set of rules applied by the medical device based on the determination. In some examples, a system comprises the computing device and the medical device.

In some examples, a method of controlling the operation of a computing device configured to communicate with a medical device configured to store episode data for episodes in response to detecting a health event of a patient, the method comprises: applying, by processing circuitry of the computing device, a second set of rules to episode data received from the medical device for each of a plurality of episodes, the episode data stored by the medical device for the episodes in response to detecting a health event based on application of a first set of rules by the medical device to sensed data sensed by the medical device; classifying, by the processing circuitry, each episode of the plurality of episodes as one of a plurality of classifications based on the application of the second set of rules to the episode data, wherein at least one of the plurality of classifications comprises a classification for which transmission of the episode data from the medical device to the computing device was unnecessary; determining, by the processing circuitry, that an amount of the plurality of episodes classified as the classification for which transmission of the episode data from the medical device to the computing device was unnecessary satisfies at least one criterion; and initiating, by the processing circuitry, a change to the first set of rules applied by the medical device based on the determination.

In some examples, a method comprises applying, by processing circuitry of at least one of a smartphone or a smartwatch configured to wirelessly communicate with an insertable cardiac monitor, a second set of rules to episode data received from the medical device for each of a plurality of episodes, the episode data stored by the medical device for the episodes in response to detecting a ventricular tachyarrhythmia based on application of a first set of rules by the medical device to sensed data sensed by the medical device, wherein the second set of rules comprises an artificial intelligence algorithm; classifying, by the processing circuitry, each episode of the plurality of episodes as one of a plurality of classifications based on the application of the second set of rules to the episode data, wherein at least one of the plurality of classifications comprises a classification for which transmission of the episode data from the medical device to the at least one of the smartphone or the smartwatch was unnecessary, the classification for which transmission of the episode data from the medical device to the at least one of the smartphone or the smartwatch was unnecessary comprises at least one of noise, oversensing, or supraventricular tachycardia; determining, by the processing circuitry, that an amount of the plurality of episodes classified as the classification for which transmission of the episode data from the medical device to the at least one of the smartphone or the smartwatch was unnecessary satisfies at least one criterion; and initiating, by the processing circuitry, a change to the first set of rules applied by the medical device based on the determination.

In some examples, a method comprises: applying, by processing circuitry of a medical device, a set of rules to data sensed by the medical device to detect a plurality of health events of a patient; for each of the plurality of health events detected by the medical device, determining, by the processing circuitry, context information for the application of the set of rules to data sensed by the medical device; and determining, by the processing circuitry, whether to at least one of store episode data for the health event in a memory of the medical device or transmit the episode data for the health event to a computing device based on the context information.

In some examples, a non-transitory computer-readable storage medium comprises instructions that, when executed by processing circuitry, cause the processing circuitry to perform any method described herein.

In some examples, a device comprises processing circuitry operably coupled to memory comprising instructions configured to cause the processing circuitry to perform any method described herein.

In some examples, a system comprises a medical device configured to sense parameter data of a patient, including at least an electrocardiogram of the patient, and processing circuitry. The processing circuitry is configured to: detect an acute health event of the patient based on application of a set of rules to a first set of the parameter data; determine that a risk of the acute health event is changed based on a second set of the parameter data; and adjust at least one of a sensitivity or specificity of the set of rules based on the determination that the risk of the acute health event is changed.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured detect acute health events of a patient, and to respond to such detections, in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a flow diagram illustrating an example operation for applying rules to patient parameter data to determine whether an acute health event is detected.

FIG. 6 is a flow diagram illustrating another example operation for applying rules to patient parameter data to determine whether an acute health event is detected.

FIG. 7 is a flow diagram illustrating an example operation for configuring rules applied to patient parameter data to determine whether an acute health event is detected for a patient.

FIG. 8 is a flow diagram illustrating another example operation for configuring rules applied to patient parameter data to determine whether an acute health event is detected for a patient.

FIG. 9 is a flow diagram illustrating an example operation for initiating a change to rules used by a medical device to identify episodes.

FIG. 10 is a flow diagram illustrating an example operation of a medical device for determining whether to store and/or transmit episode data.

FIG. 11 is a conceptual diagram illustrating an example machine learning model configured to determine an extent to which data of a patient indicates an acute health event.

FIG. 12 is a conceptual diagram illustrating an example training process for an artificial intelligence model, in accordance with examples of the current disclosure.

FIG. 13A is a perspective drawing illustrating an insertable cardiac monitor.

FIG. 13B is a perspective drawing illustrating another insertable cardiac monitor.

Like reference characters refer to like elements throughout the figures and description.

### DETAILED DESCRIPTION

A variety of types of implantable and external devices are configured to detect arrhythmia episodes and other acute health events based on sensed ECGs and, in some cases, other physiological signals. External devices that may be used to non-invasively sense and monitor ECGs and other physiological signals include wearable devices with electrodes configured to contact the skin of the patient, such as patches, watches, rings, necklaces, hearing aids, clothing, car seats, or bed linens. Such external devices may facilitate relatively longer-term monitoring of patient health during normal daily activities.

Implantable medical devices (IMDs) also sense and monitor ECGs and other physiological signals, and detect acute health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ II™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

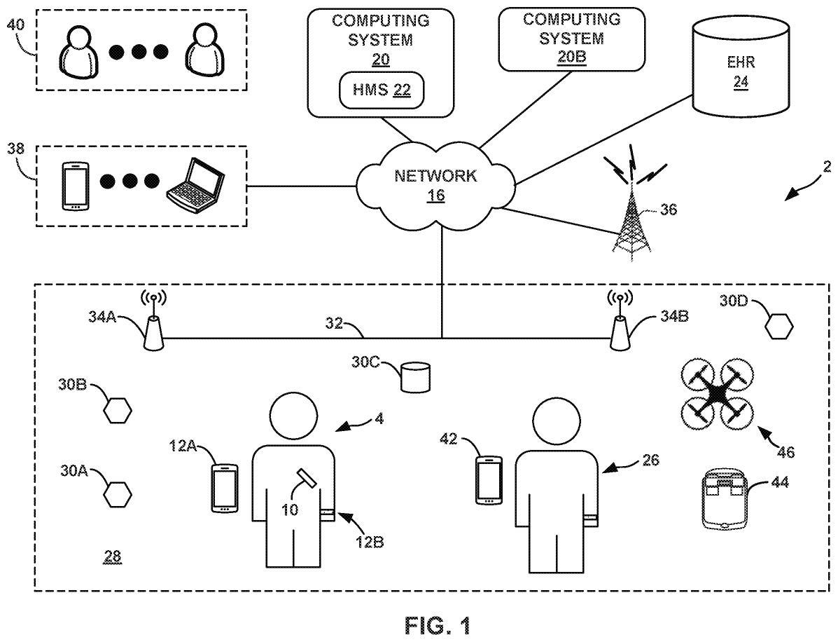

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. The example techniques may be used with one or more patient sensing devices, e.g., IMD 10, which may be in wireless communication with one or more patient computing devices, e.g., patient computing devices 12A and 12B (collectively, “patient computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ II™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4. For example, a system with two IMDs 10 may capture different values of a common patient parameter with different resolution/accuracy based on their respective locations. In some examples, instead of or in addition to two IMDs 10, system 2 may include a ventricular assist device or WAED in addition to IMD 10.

Patient computing devices 12 are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing devices 12 may communicate with IMD 10 and each other according to the Bluetooth® or Bluetooth® Low Energy (BLE) protocols, as examples. In some examples, only one of computing devices 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilities detection of acute health events of patient 4 by system 2, and the responses of system 2 to such acute health events.

Computing device(s) 12 may transmit data, including data retrieved from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to detect acute health events of patient 4, such as SCA, based on data sensed by IMD 10 and, in some cases, other data, such as data sensed by computing devices 12A and/or 12B, and data from EHR 24. To detect acute health events, IMD 10 may apply rules to the data, which may be referred to as patient parameter data. In response to detection of an acute health event, IMD 10 may wirelessly transmit a message to one or both of computing devices 12A and 12B. The message may indicate that IMD 10 detected an acute health event of the patient. The message may indicate a time that IMD 10 detected the acute health event. The message may include physiological data collected by IMD 10, e.g., data which lead to detection of the acute health event, data prior to detection of the acute health event, and/or real-time or more recent data collected after detection of the acute health event. The physiological data may include values of one or more physiological parameters and/or digitized physiological signals. Examples of acute health events are SCA, a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

In some examples, the detection of the acute health event by IMD 10 may include multiple phases. For example, IMD 10 may complete an initial detection of the acute health event, e.g., SCA or tachyarrhythmia, and initiate wireless communication, e.g., Bluetooth® or Bluetooth Low Energy®, with computing device(s) 12 in response to the initial detection. The initial detection may occur five to ten seconds after onset of the acute health event, for example. IMD 10 may continue monitoring to determine whether the acute health event is sustained, e.g., a sustained detection of SCA or tachyarrhythmia. In some examples, IMD 10 may use more patient parameters and/or different rules to determine whether event is sustained or otherwise confirm detection.

Initiating communication with computing device(s) 12 in response to an initial detection may facilitate the communication being established at the time the acute health event is confirmed as sustained. To conserve power of IMD 10 and computing device(s) 12, IMD 10 may wait to send the message, e.g., including sensed data associated with the acute health event, until it is confirmed as sustained, which may be determined about thirty seconds after onset of the event, or after a longer period of time. Less urgent events may have longer confirmation phases and may be alerted with less urgency, such being alerted as health care events rather than acute health events. However, the initiation of communication after initial detection may still benefit less urgent events. Conserving power may be significant in the case of non-rechargeable IMDs to prolong their life prior to needing surgery for replacement, as well as for rechargeable IMDs or external devices to reduce recharge frequency.

In response to the message from IMD 10, computing device(s) 12 may output an alarm that may be visual and/or audible, and configured to immediately attract the attention of patient 4 or any person in environment 28 with patient 4, e.g., a bystander 26. Additionally or alternatively, computing device(s) 12 may transmit an alert or alarm message to devices and users outside the visible/audio range of computing device(s) 12, e.g., to IoT devices 30, bystander computing device 42, or HMS 22. Environment 28 may be a home, office, or place of business, or public venue, as examples. An alert or alarm message sent to HMS 22 via network 16, or other messages sent by computing device(s) 12, may include the data received from IMD 10 and, in some cases, additional data collected by computing device(s) 12 or other devices in response to the detection of the acute health event by IMD 10. For example, the message may include a location of patient 4 determined by computing device(s) 12. In some examples, computing device(s) 12 may further configure or change the content of alert or alarm messages based on the location of patient 4, e.g., different messages may be sent depending on whether patient 4 is at home, another residence, an office or business, a public location, or in a health care facility. The health care needed by patient, and thus the messaging of system 2, may vary depending on the location of patient 4.

Other devices in the environment 28 of patient 4 may also be configured to output alarms or take other actions to attract the attention of patient 4 and, possibly, a bystander 26, or to otherwise facilitate the delivery of care to patient 4. For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, televisions, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras, microphones, or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of an acute health event when communication with computing devices 12 is unavailable. In such examples, IoT device(s) 30 may be configured to provide some or all of the functionality ascribed to computing devices 12 herein.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing devices 12, IoT devices 30, and other devices within environment 28 may be configured to communicate with network 16, e.g., with HMS 22, via a cellular base station 36 and a cellular network.

Computing device(s) 12, and in some examples IoT device(s) 30, may include input devices and interfaces to allow a user to override the alarm in the event the detection of the acute health event by IMD 10 was false. In some examples, one or more of computing device(s) 12 and IoT device(s) 30 may implement an event assistant. The event assistant may provide a conversational interface for patient 4 and/or bystander 26 to exchange information with the computing device or IoT device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user. For example, patient 4 may indicate that they feel dizzy and ask the event assistant, “how am I doing?”.

In some examples, computing device(s) 12 and/or HMS 22 may implement one or more algorithms to evaluate the sensed physiological data received from IMD 10, and in some cases additional physiological or other patient parameter data sensed or otherwise collected by the computing device(s) or IoT devices 30, to confirm or override the detection of the acute health event by IMD 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event.

In examples in which computing device(s) 12 are configured perform an acute health event confirmation analysis, computing device(s) 12 may transmit alert messages to HMS 22 and/or IoT devices 30 in response to confirming the acute health event. In some examples, computing device(s) 12 may be configured to transmit the alert messages prior to completing the confirmation analysis, and transmit cancellation messages in response to the analysis overriding the detection of the acute health event by IMD 10. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30.

For example, HMS 22 may be configured to transmit alert messages to one or computing devices 38 associated with one or more care providers 40 via network 16. Care providers may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, and IoT device(s) 30, including sensed physiological data, time of the acute health event, location of patient 4, and results of the analysis by IMD 10, computing device(s) 12, IoT device(s) 30, and/or HMS 22. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or IoT devices 30 may be configured to automatically contact EMS, e.g., autodial 911, in response to receiving an alert message from IMD 10. Again, such operations may be cancelled by patient 4, bystander 26, or another user via a user interface of computing device(s) 12 or IoT device(s) 30, or automatically cancelled by computing device(s) 12 based on a confirmatory analysis performed by the computing device(s) overriding the detection of the acute health event by IMD 10.

Similarly, HMS 22 may be configured to transmit an alert message to computing device 42 of bystander 26, which may improve the timeliness and effectiveness of treatment of the acute health event of patient 4 by bystander 26. Computing device 42 may be similar to computing devices 12 and computing devices 38, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36, using any of the networking methods described herein.

In some examples, the alert message to bystander 26 may be configured to assist a layperson in treating patient. For example, the alert message to bystander 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an automated external defibrillator (AED) 44 or life vest, and instructions for use of the equipment. In some examples, computing device(s) 12, IoT device(s) 30, and/or computing device 42 may implement an event assistant configured to use natural language processing and context data to provide a conversational interface for bystander 42. The assistant may provide bystander 26 with directions for providing care to patient 4, and respond to queries from bystander 26 about how to provide care to patient 4.

In some examples, HMS 22 may mediate bi-directional audio (and in some cases video) communication between care providers 40 and patient 4 or bystander 26. Such communication may allow care providers 40 to evaluate the condition of patient 4, e.g., through communication with patient 4 or bystander 26, or through use of a camera or other sensors of the computing device or IoT device, in advance of the time they will begin caring for the patient, which may improve the efficacy of care delivered to the patient. Such communication may also allow the care providers to instruct bystander 42 regarding first responder treatment of patient 4.

In some examples, HMS 22 may control dispatch of a drone 46 to environment 28, or a location near environment 28 or patient 4. Drone 46 may be a robot and/or unmanned aerial vehicle (UAV). Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4.

Any of IMD 10, computing device(s) 12, IoT device(s) 30, computing device(s) 38 and 42, AED 44, drone 46, or HMS 22 may, individually or in any combination, perform the operations described herein for detection of acute health events, such as SCA, by applying rules, which may include one or more machine learning models, to patient parameter data to detect acute health events. For example, one of these devices, or more than one of them in cooperation, may apply a first set of rules to a first set of patient parameter data for a first determination of whether an acute health event is detected and, based on whether one or more context criteria associated with the first determination are satisfied, determine whether to apply a second set of rules to second patient parameter data to determine whether the acute health event is detected.

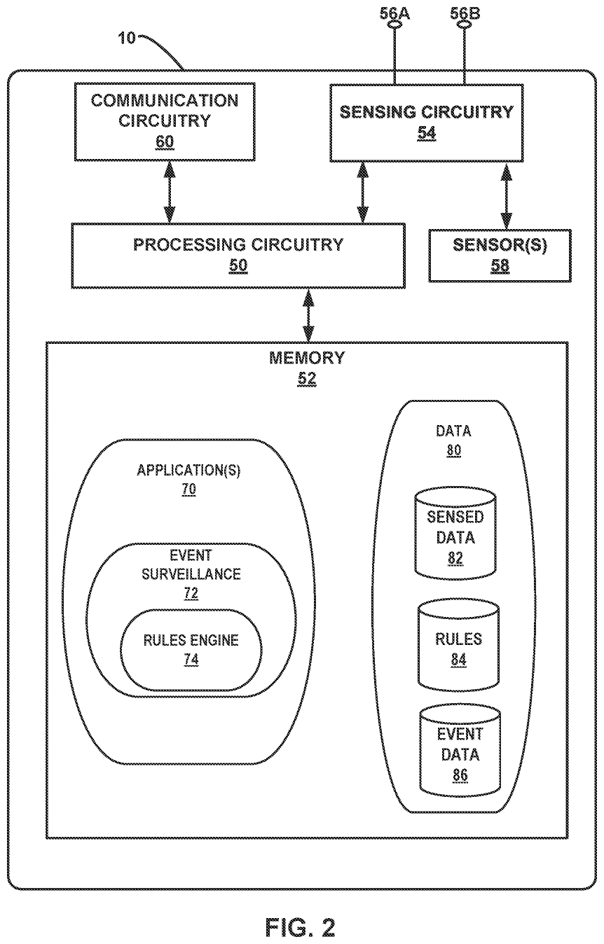

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, T-wave alternans, intra-beat intervals (e.g., QT intervals), and/or ECG morphologic features, to detect an episode of cardiac arrhythmia of patient 4. Example Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration, cardiac pulse or flow, and a degree of perfusion or edema. Processing circuitry 50 may determine physiological data relating to respiration, cardiac pulse or flow, perfusion, and/or edema based on the measured impedance.

In some examples, IMD 10 includes one or more sensors 58, such as one or more accelerometers, gyroscopes, microphones, optical sensors, temperature sensors, pressure sensors, and/or chemical sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52. Patient parameters determined from signals from sensors 58 may include oxygen saturation, glucose level, stress hormone level, heart sounds, body motion, body posture, or blood pressure.

Memory 52 may store applications 70 executable by processing circuitry 50, and data 80. Applications 70 may include an acute health event surveillance application 72. Processing circuitry 50 may execute event surveillance application 72 to detect an acute health event of patient 4 based on combination of one or more of the types of physiological data described herein, which may be stored as sensed data 82. In some examples, sensed data 82 may additionally include patient parameter data sensed by other devices, e.g., computing device(s) 12 or IoT device(s) 30, and received via communication circuitry 60. Event surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning, e.g., may include one or more machine learning models.

As examples, event surveillance application 72 may detect SCA, a ventricular fibrillation, a ventricular tachycardia, supra-ventricular tachycardia (e.g., conducted atrial fibrillation), ventricular asystole, or a myocardial infarction based on an ECG and/or other patient parameter data indicating the electrical or mechanical activity of the heart of patient 4. In some examples, event surveillance application 72 may detect stroke based on such cardiac activity data. In some examples, sensing circuitry 54 may detect brain activity data, e.g., an electroencephalogram (EEG) via electrodes 56, and event surveillance application 72 may detect stroke or a seizure based on the brain activity alone, or in combination with cardiac activity data or other physiological data. In some examples, event surveillance application 72 detects whether the patient has fallen based on data from an accelerometer alone, or in combination with other physiological data. When event surveillance application 72 detects an acute health event, event surveillance application 72 may store the sensed data 82 that lead to the detection (and in some cases a window of data preceding and/or following the detection) as event data 86.

In some examples, in response to detection of an acute health event, processing circuitry 50 transmits, via communication circuitry 60, event data 86 for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

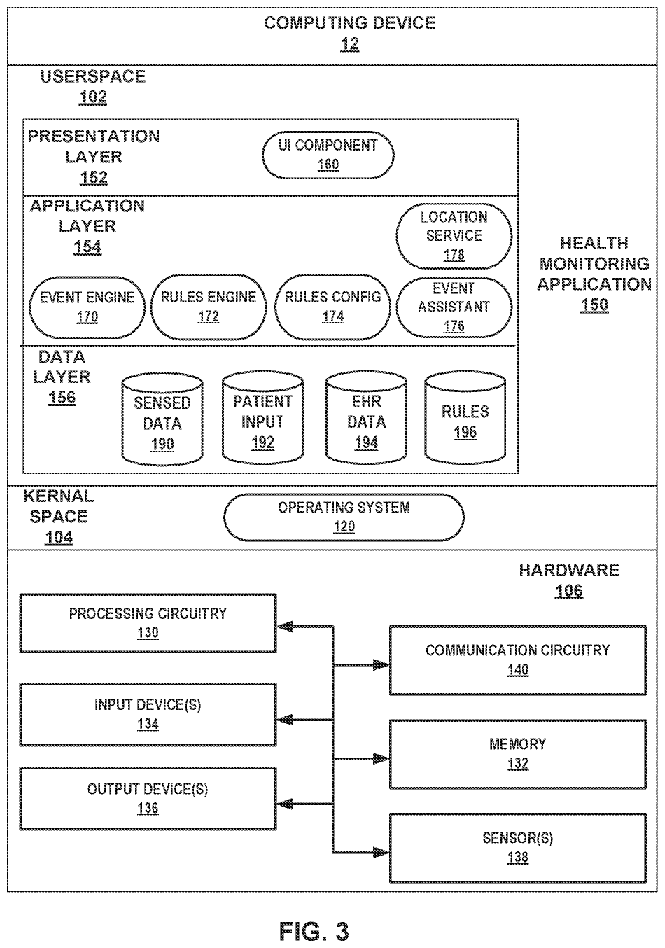

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30, computing devices 38 and 42, AED 44, and/or drone 46 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICS, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, haptic, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers (e.g., 3-axis accelerometers), an optical sensor, impedance sensors, temperature sensors, pressure sensors, heart sound sensors (e.g., microphones), and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 172 may be responsive to receipt of an alert transmission from IMD 10 indicating that IMD 10 detected an acute health event. Event engine 172 may control performance of any of the operations in response to detection of an acute health event ascribed herein to computing device 12, such as activating an alarm, transmitting alert messages to HMS 22, controlling IoT devices 30, and analyzing data to confirm or override the detection of the acute health event by IMD 10.

Rules engine 174 analyzes sensed data 190, and in some examples, patient input 192 and/or EHR data 194, to determine whether there is a sufficient likelihood that patient 4 is experiencing the acute health event detected by IMD 10. Sensed data 190 may include data received from IMD 10 as part of the alert transmission, additional data transmitted from IMD 10, e.g., in “real-time,” and physiological and other data related to the condition of patient 4 collected by, for example, computing device(s) 12 and/or IoT devices 30. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystander 26. The queries and responses may occur responsive to the detection of the event by IMD 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of SCA, tachyarrhythmia, myocardial infarction, stroke, seizure, one or more disease states, such as status of heart failure chronic obstructive pulmonary disease (COPD), renal dysfunction, or hypertension, aspects of disease state, such as ECG characteristics, cardiac ischemia, oxygen saturation, lung fluid, activity, or metabolite level, genetic conditions, congenital anomalies, history of procedures, such as ablation or cardioversion, and healthcare utilization. EHR data 194 may also include cardiac indicators, such as ejection fraction and left-ventricular wall thickness. EHR data 194 may also include demographic and other information of patient 4, such as age, gender, race, height, weight, and BMI.

Rules engine 172 may apply rules 196 to the data. Rules 196 may include one or more models, algorithms, decision trees, and/or thresholds. In some cases, rules 196 may be developed based on machine learning, e.g., may include one or more machine learning models. In some examples, rules 196 and the operation of rules engine 172 may provide a more complex analysis the patient parameter data, e.g., the data received from IMD 10, than is provided by rules engine 74 and rules 84. In examples in which rules 196 include one or more machine learning models, rules engine 172 may apply feature vectors derived from the data to the model(s).

Rules configuration component 174 may be configured to modify rules 196 (and in some examples rules 84) based on feedback indicating whether the detections and confirmations of acute health events by IMD 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

Rules configuration component 174, or another component executed by processing circuitry of system 2, may select a configuration of rules 196 based on etiological data for patient, e.g., any combination of one or more of the examples of sensed data 190, patient input 192, and EHR data 194 discussed above. In some examples, different sets of rules 196 tailored to different cohorts of patients may be available for selection for patient 4 based on such etiological data.

As discussed above, event assistant 176 may provide a conversational interface for patient 4 and/or bystander 26 to exchange information with computing device 12. Event assistant 176 may query the user regarding the condition of patient 4 in response to receiving the alert message from IMD 10. Responses from the user may be included as patient input 192. Event assistant 176 may use natural language processing and context data to interpret utterances by the user. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystander 26.

Location service 178 may determine the location of computing device 12 and, thereby, the presumed location of patient 4. Location service 178 may use global position system (GPS) data, multilateration, and/or any other known techniques for locating computing devices.

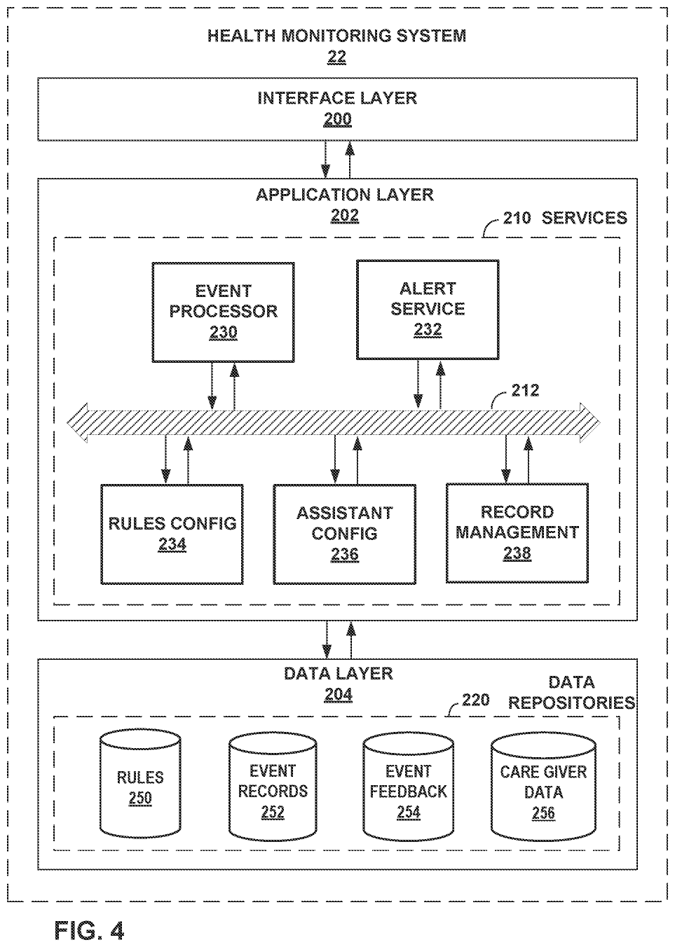

FIG. 4 is a block diagram illustrating an operating perspective of HMS 22. HMS 22 may be implemented in a computing system 20, which may include hardware components such as those of computing device 12, e.g., processing circuitry, memory, and communication circuitry, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, bystander 26, and care providers 40, activating drone 46 and, in some cases, analyzing data to confirm or override the detection of the acute health event by IMD 10.

Record management service 238 may store the patient data included in a received alert message within event records 252. Alert service 232 may package the some or all of the data from the event record, in some cases with additional information as described herein, into one more alert messages for transmission to bystander 26 and/or care providers 40. Care giver data 256 may store data used by alert service 232 to identify to whom to send alerts based on locations of potential bystanders 26 and care givers 40 relative to a location of patient 4 and/or applicability of the care provided by care givers 40 to the acute health event experienced by patient 4.

In examples in which HMS 22 performs an analysis to confirm or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMD 10. In such examples, rules configuration service 250 may be configured to develop and maintain rules 196 and rules 84. Rules configuration service 234 may be configured to develop different sets of rules 84, 196, 250, e.g., different machine learning models, for different cohorts of patients. Rules configuration service 234 may be configured to modify these rules based on event feedback data 254 that indicates whether the detections and confirmations of acute health events by IMD 10, computing device 12, and/or HMS 22 were accurate. Event feedback 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices. For example, assistant configuration service 236 may provide event assistants updates to their natural language processing and context analyses to improve their operation over time. In some examples, assistant configuration service 236 may apply machine learning techniques to analyze sensed data and event assistant interactions stored in event records 252, as well as the ultimate disposition of the event, e.g., indicated by EHR 24, to modify the operation of event assistants, e.g., for patient 4, a class of patients, all patients, or for particular users or devices, e.g., care givers, bystanders, etc.

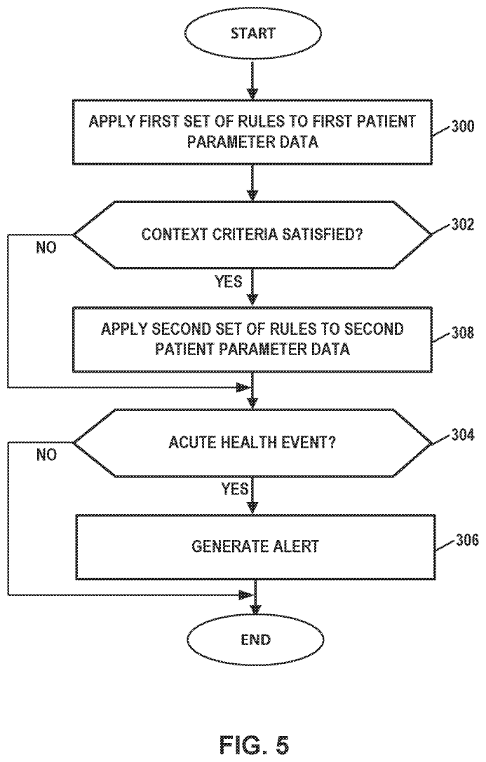

FIG. 5 is a flow diagram illustrating an example operation for applying rules to patient parameter data to determine whether an acute health event is detected. The example operation of FIG. 5 may be performed by processing circuitry of any one of IMD 10, computing device(s) 12, 38, 42, IoT devices 30, AED 44, drone 46, or HMS 22 (e.g., by processing circuitry 50 or 130 implementing rules engine 74 or 172 and applying rules 84 or 196), or by processing circuitry of two or more of these devices respectively performing portions of the example operation.

According to the example of FIG. 5, the processing circuitry applies a first set of rules to first patient parameter data for a first determination of whether an acute health event, e.g., SCA, is detected (300). The processing circuitry determines whether one or more context criteria associated with the first determination are satisfied (302). If the one or more context criteria are not satisfied (NO of 302), the processing circuitry may determine whether the acute health event is detected based on the first determination (304). If the acute health event is detected (YES of 304), the processing circuitry may generate an alert, e.g., a message to another device and/or a user-perceptible alert as described herein (306). If the acute health event is not detected (NO of 304) or the alert has been generated, the example operation of FIG. 5 may end. If the one or more context criteria are satisfied (YES of 302), the processing circuitry may apply a second set of rules to second patient parameter data for a second determination of whether the acute health event, e.g., SCA, is detected (308), and determine whether the acute health event is detected based on the second determination (304).

The first and second sets of rules are different in at least one aspect. In some examples, the second set of rules comprises at least one machine learning model. In some examples, both the first and second sets of rules comprise at least one machine learning model.

In some examples, the processing circuitry determines a risk score of the acute health event, e.g., SCA, based on the application of the first set of rules to the first patient parameter data, and compares the risk score to a threshold to determine whether the one or more context criteria are satisfied. In some examples, the context indicating that the second set of rules should be applied to the second patient parameter data may be that the risk score produced by the first determination does not meet a threshold indicating a sufficient certainty that the acute health event is occurring. The risk score may be a percentage likelihood of the acute health event.

In some examples, the processing circuitry determines a confidence level of the first determination of whether the acute health event is detected, and compares the confidence level to a threshold. In some examples, the one or more context criteria may be satisfied where the first determination does not have a threshold degree of confidence, or where the first determination is associated with a likelihood of being a false positive that exceeds a threshold. In such examples, application of the second set of rules to the second patient parameter data may act as a “tie-breaker” when the first determination is not confident. In some examples, the processing circuitry determines that the one or more context criteria are satisfied when input from a user, e.g., the patient, contradicts the first determination (e.g., that the acute health event was detected or not detected), indicating that the likelihood that the first determination is false may be relatively high.

The processing circuitry may determine a confidence level of the first determination of whether the acute health event is present using a variety of techniques. For example, the application of the first set of rules to the first patient parameter data may produce a level of confidence through its output, e.g., a risk score. In such examples, a higher output indicating a higher likelihood of the acute health event may indicate a higher level of confidence. Examples of rules that may produce such outputs include machine learning models and time-domain signal processing algorithms.

In some examples, the processing circuitry may determine a noise level of one or more signals from which the first patient parameter data is determined. In such examples, the processing circuitry may determine a confidence level of the first determination of whether the acute health event is present based on a noise level. In general, confidence level and noise level may be inversely related. In some examples, the processing circuitry may determine the confidence level based on health record data for patient 4. For example, if a clinician has indicated in a health record or via programming IMD 10 that patient 4 has experienced a myocardial infarction or has heart failure, confidence levels may be increased and/or thresholds included in the rules applied to the first patient parameter data may be lowered.

In some examples, a context criterion may be satisfied when a component of system 2, e.g., IMD 10 or computing devices 12, has sufficient power to enable the application of the second set of rules to the second patient parameter data. In some examples, to determine whether the one or more context criteria are satisfied, the processing may determine a power level of system 2, e.g., of the relevant device, and compare the power level threshold. In some examples, the second patient parameter data includes data of at least one patient parameter that is not included in the first patient parameter data. In some examples, the processing circuitry activates a sensor to sense this patient parameter, e.g., when the device including the sensor has sufficient power for the measurement.

In some examples, the first patient parameter data and the second patient parameter data are both sensed by an implantable medical device. In some examples, the at least one patient parameter that is included in the second patient parameter data but not included in the first patient parameter data is sensed by an external device. In some examples, processing circuitry 50 of IMD 10 or processing circuitry 130 of computing device(s) 12 (or IoT devices 30 or the other devices discussed herein) performs each of sub-operations 300-308. In other examples, processing circuitry 50 of IMD 10 performs the first determination of whether the acute health event, e.g., SCA, is detected (300), and processing circuitry 130 of computing device(s) 12 (or IoT devices 30 or the other devices discussed herein) performs each of sub-operations 302-308.

In some examples, the first patient parameter data includes at least one patient parameter determined from ECG data, and the at least one patient parameter comprises a patient parameter determined from at least one of heart sounds of the patient, an impedance of the patient, motion of the patient, respiration of the patient, posture of the patient, blood pressure of the patient, a chemical detected in the patient, or an optical signal from the patient. In some examples, the first patient parameter data and second patient parameter data may be determined using different combinations of sensors, e.g., internal and/or external sensors. The first and second determinations may be considered different tiers, with the second determination utilizing additional sensor(s), data, and/or power if the context suggests it would be desirable to supplement the first determination.

In some examples, the processing circuitry selects at least one of the second set of rules or the parameters used for the second patient parameter data based on at least one of user (e.g., patient or care giver or bystander) input or medical record information of the patient. In some examples, the user input and/or medical history information may include information entered by a clinician when programming IMD 10. For example, the processing circuitry may select at least one of the second set of rules or the parameters used for the second patient parameter data based on user input or medical record information indicating a particular symptom or condition of the patient. In some examples, the first patient parameter data comprises data for a first set of patient parameters, and the processing circuitry may select at least one of the second set of rules or a second patient parameter for the second patient parameter data based on the level. A level for a particular parameter that is clinically significant but contrary to the first determination (either a detection or non-detection), may suggest that the second determination should be performed, and should be performed with a particular parallel (but different) or orthogonal patient parameter to resolve the uncertainty about whether the acute health event is detected.

In some examples, the first patient parameter data includes at least one patient parameter determined from ECG data of the patient, and the second patient parameter data comprises at least one of a morphological change or a frequency shift of the ECG data over time. The processing circuitry may analyze ECG data for timing or morphology changes. For example, morphological or frequency changes as a ventricular fibrillation persists may indicate an increase lethality of the ventricular fibrillation. In some examples, the rules applied processing circuitry may determine a higher likelihood of the acute health event, e.g., lethal ventricular fibrillation or SCA, the presence of such morphological or frequency shifts.

The example operation of FIG. 5 may result in a hierarchy of rules or even sensor measurements. In some examples, one or more sensors may be activated in certain contexts, and may be inactive for first determinations of whether the acute health event is detected, e.g., to conserve power of IMD 10. For example, if in a first determination ECG data indicates ventricular fibrillation and other sensor data indicates no pulse and no heart sounds, there may be no need for the second determination. But if those levels of evidence are not high, e.g., not sure if it definitely ventricular fibrillation there might be faint heart sounds or faint pulses, then a second determination could be employed.

Further, the rules and sensors used in either or both of the first as second determinations can be configured/personalized for each patient based on their medical history from EMR or their history of previous events or by their physicians/caregivers depending on the situation. For example, if a caregiver has to leave town for few days, the processing circuitry could configure the rules to be satisfied by lower levels of evidence, e.g., automatically, which may advantageously tailor the monitoring provided by system 2 to the context of patient 4 and care givers of the patient.

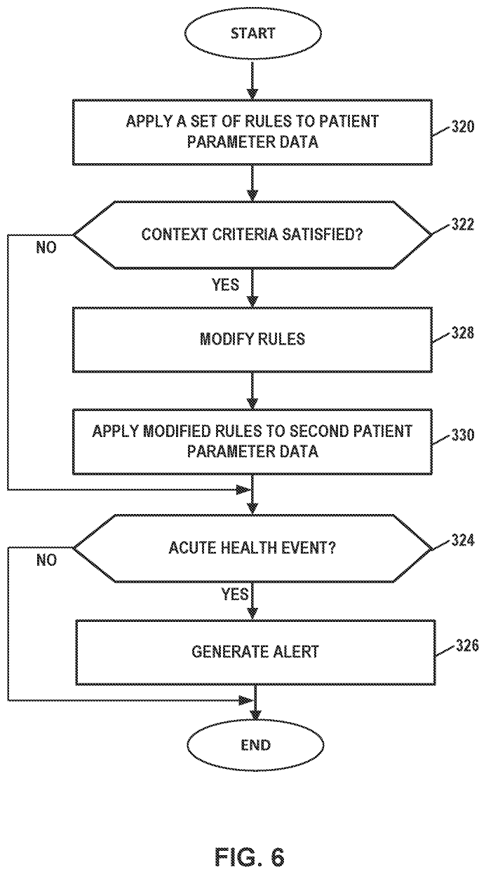

FIG. 6 is a flow diagram illustrating another example operation for applying rules to patient parameter data to determine whether an acute health event is detected. The example operation of FIG. 6 may be performed by processing circuitry of any one of IMD 10, computing device(s) 12, 38, 42, IoT devices 30, AED 44, drone 46, or HMS 22 (e.g., by processing circuitry 50 or 130 implementing rules engine 74 or 172 and applying rules 84 or 196), or by processing circuitry of two or more of these devices respectively performing portions of the example operation.

According to the example of FIG. 6, the processing circuitry applies a set of rules to patient parameter data to determine whether an acute health event, e.g., SCA, is detected (320). The processing circuitry determines whether one or more context criteria associated with the determination are satisfied (322). If the one or more context criteria are not satisfied (NO of 322), the processing circuitry may determine whether the acute health event is detected based on the determination (324). If the acute health event is detected (YES of 324), the processing circuitry may generate an alert, e.g., a message to another device and/or a user-perceptible alert as described herein (326). If the acute health event is not detected (NO of 324) or the alert has been generated, the example operation of FIG. 6 may end. If the one or more context criteria are satisfied (YES of 322), the processing circuitry may modify the set of rules (328), apply second patient parameter data to the second set of rules (330), and determine whether the acute health event is detected based on the application of the second patient parameter data to the second set of rules (324).

The processing circuitry may determine whether the one or more context criteria are satisfied in the manner described with respect to FIG. 5. In some examples, the first and second patient parameter data may be determined from the same patient parameters or (with respect to at least one parameter) different patient parameters. In some examples, the first patient parameter data and the second patient parameter data include at least one common patient parameter, and the processing circuitry may change a mode sensing for the common patient parameter between the first patient parameter data and the second patient parameter data in response to satisfaction of the one or more context criteria. For example, the processing circuitry may change a sampling frequency for the common patient parameter.

In some examples in which IMD 10 senses patient parameters used to determine the first patient parameter data, the processing circuitry may determine that a context criterion is satisfied by detecting that IMD 10 has flipped or otherwise migrated within patient 4. Such migration may lead to significant changes in patient parameter data, e.g., ECG data, impedance data, or heart sound data. Changing a mode employed by IMD 10 to sense one or more patient parameters, or changing rules to account for changes in patient parameter data resulting from device migration, may help ameliorate the device migration and maintain effective acute health event detection. In addition to the mode of sensing and/or rules, the processing circuitry may adjust other aspects of system, such mode of wireless communication between the IMD and other devices. Techniques for detecting and mitigating migration of IMD 10 are described in commonly-assigned U.S. patent application Ser. No. 17/101,945, filed Nov. 23, 2020 by Anderson et al., titled “DETECTION AND MITIGATION OF INACCURATE SENSING BY AN IMPLANTED SENSOR OF A MEDICAL SYSTEM,” which is incorporated herein by reference in its entirety.

In some examples, the processing circuitry determines that the one or more context criteria are satisfied when the processing circuitry determines that the acute health event, e.g., ventricular tachyarrhythmia or SCA, is detected, but the patient or another user cancels the alarm or otherwise provides user input contradicting the determination. In such examples, the processing circuitry may modify one or both of the sensed patient parameters or the rules applied to the patient parameter data.

For example, the patient may have tolerated a rapid ventricular tachycardia that lasted for a sustained period (e.g., a programmed 10 or 20 seconds), but could experience another arrhythmia, e.g., syncope, soon even though the patient believes they are OK. The modification may include adapting the rules based on the rhythm. Sometimes a long duration episode accelerates to ventricular fibrillation or more rapid ventricular tachycardia. Sometimes ventricular fibrillation slows down. In either case, the modification could include changing a heart rate threshold, e.g., applying hysteresis to the heart rate threshold. In some examples, ventricular fibrillation becomes ugly/fine and is very difficult to sense. In such examples, the modification may include changing a ventricular depolarization detection threshold to allow more undersensing of depolarizations.

In some examples, the processing circuitry determines that the one or more context criteria are satisfied based on a recent history of high arrhythmia burden. Some patients have electrical storms. Their electrolytes may be imbalanced, and they may experience a cluster of ventricular arrhythmias, but the patient parameter data may not satisfy the rules for detection of the acute health event. In such cases, the processing circuitry may adapt a tachyarrhythmia duration the threshold, may alert patient and caregivers and inform them to seek care ASAP, and/or may alert a clinician and send patient parameter data, e.g., ECG data, so the clinician can review.

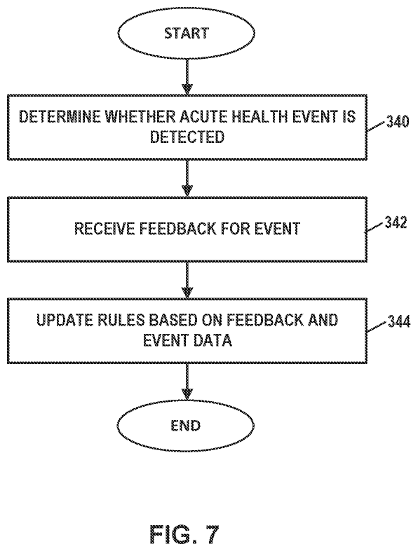

FIG. 7 is a flow diagram illustrating an example operation for configuring rules applied to patient parameter data to determine whether an acute health event is detected for a patient. The example operation of FIG. 7 may be performed by processing circuitry that implements HMS 22, e.g., that implements rules configuration service 234. In some examples, the operation of FIG. 7 may be performed by processing circuitry of any one of IMD 10, computing device(s) 12, 38, 42, IoT devices 30, AED 44, drone 46, or HMS 22, e.g., implementing a rules configuration module, or by processing circuitry of two or more of these devices respectively performing portions of the example operation.

According to the example operation of FIG. 7, the processing circuitry determines whether an acute health event, e.g., SCA, is detected (340). The processing circuitry receives feedback for the event (342). The feedback indicates whether the detection is a true or false positive, or the non-detection is a true or false negative. The processing circuitry may receive the feedback from patient 4, care giver 40, bystander 26, or EHR 24. The processing circuitry updates rules (e.g., rules 84, rules 196, and/or rules 250) based on the feedback and event data, e.g., event data 86 or event records 252. In some examples, uses the event data as a training set for one or more machine learning models based on the feedback.

Through predictive and “self-learning” techniques, the operation of a system used to provide an alert for SCA can be improved. Time-to-treatment (either CPR or a shock from AED 44) may be improved by providing a timely alert, either to bystanders 26 or the EMS care givers 40. The information used to improve the performance could include physiologic sensor data that may indicate an SCA event is likely (QT prolongation, T-wave alternans, changes in respiration rate or thoracic impedance, etc.). The information used to improve the performance could include information indicating whether the prior SCA event was alerted appropriately and accurately, clinical or physiologic characteristics of the patient (disease state, weight, gender, etc.), data from EHR 24, and data input from the patient (e.g., symptom logging, confirmation that he/she is OK and not suffering from SCA, etc.).

Implementing the example operation of FIG. 7, the processing circuitry may personalize the rules for patient 4 over time. If patient 4 has a lot of false positives, the example operation of FIG. 7 may modify the rules to be less sensitive and, conversely, if the patient 4 has a lot of false negatives may modify the rules to be more sensitive. In some examples, the processing circuitry may use the feedback and event data to update rules, e.g., machine learning models, for other patients, such as all patients whose IMDs are served by EMS 22, or a particular population or cohort of patients. In some examples, the processing circuitry may use data from a number of sources (e.g., computing devices 12, IoT devices 30, AED 44, or drone 46) to modify the rules or the collection of patient parameter data. Data used by processing circuitry to update rules may include data indicating a duration of CPR, e.g., input by a user, or data collected using an accelerometer, speaker, light detector, or camera on a computing device or IoT device.

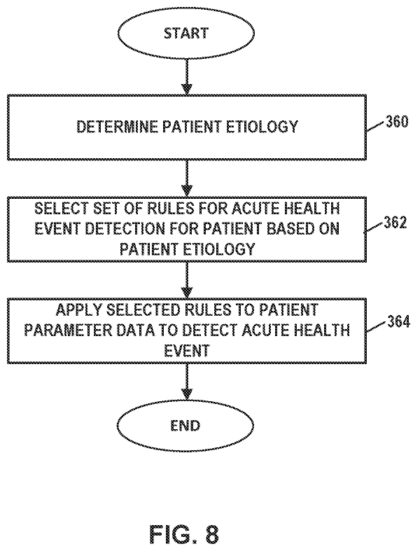

FIG. 8 is a flow diagram illustrating another example operation for configuring rules applied to patient parameter data to determine whether an acute health event is detected for a patient. The example operation of FIG. 8 may be performed by processing circuitry that implements HMS 22, e.g., that implements rules configuration service 234. In some examples, the operation of FIG. 8 may be performed by processing circuitry of any one of IMD 10, computing device(s) 12, 38, 42, IoT devices 30, AED 44, drone 46, or HMS 22, e.g., implementing a rules configuration module, or by processing circuitry of two or more of these devices respectively performing portions of the example operation.

According to the example operation of FIG. 8, the processing circuitry determines an etiology or risk stratification of patient 4 (360). The processing circuitry selects a set of rules (e.g., a set of rules 84, rules 196, and/or rules 250), which may be a first set of rules and/or a second set of rules, for acute health event, e.g., SCA, detection for patient 4 based on the patient etiology (362). In some examples, rules 250 include different sets of rules for different patient cohorts having different etiologies, and processing circuitry may select different rule sets to implement as rules 84 in IMD 10 and rules 196 in computing device(s) 12 for a given patient based on the etiology of that patient. The processing circuitry may apply the selected set of rules to patient parameter data to determine whether the acute health event is detected using any of the techniques described herein (364).

Detection of SCA can be achieved by looking at a number of possible markers that occur prior to and during the event. The best markers to detect an impending or ongoing event are likely to be different based on an etiology of the patient. An SCA detection algorithm which uses a generic algorithm designed for a broad population may not achieve satisfactory sensitivity and specificity. The etiology of patient 4 may include baseline characteristics, medical history, or disease state. The etiology of patient 4 may include any EHR data 194 described herein, as well as patient activity level or metabolite level. With such possible inputs, the rules could look for certain markers to exhibit certain trends or threshold crossings to detect an impending or ongoing acute health event, e.g., SCA.

In some examples, selection of a set of rules may include modification of a universal rule set to turn certain rules (or markers of the acute health event) on or off, or change the weight of certain rules or markers. In some examples, a family of devices could be designed such that individual models have sensors or calculation for only a limited set of inputs motivated by a need to reduce manufacturing costs or energy consumption.

While SCA is typically detected by heart rate/rhythm, rules related to other patient parameter data may be set to a heightened alert based patient etiology. For example, a patient with prior myocardial infarction may have rules that weigh ischemia factors such as ST segment elevation more heavily than for patients lacking this etiology. As another example, a patient with long QT syndrome may have rules that more heavily weight QT interval and activity. As another example, rules for a heart failure patient may have rules that apply greater weight to patient parameter data related to lung fluid and QRS duration.

In some examples, processing circuitry of system 2 may use patient etiology to “personalize” other aspects of the operation of system 2 for patient 4 or a cohort including patient 4. For example, the processing circuitry may provide alerts and user interfaces that guide care givers 40, bystanders 26, patient 4, or others based on the etiology. The processing circuitry can provide patient-specific care recommendations (e.g., AED or potential drug therapy for prevention or therapy of SCA). The ability of the system to detect the acute health event with adequate sensitivity and specificity may, for example, guide an EMS care giver 40 to what they can expect when they arrive on the scene and how best to treat the presenting rhythm, e.g., is the patient hypoxic, hypovolemic, hypothermic, tension pneumothorax, cardiac tamponade (the H's and T's of Advanced Cardiac Life Support). The etiology may indicate of patient 4 is more at risk for pulseless electrical activity vs. ventricular fibrillation/ventricular tachycardia. The processing circuitry of system 2 may provide care givers information based on the etiology current patient parameter data of patient 4, such as recommendations to provide CPR or defibrillation, provide drugs, or induce hypothermia. The processing circuitry of system 2 may recommend patient-specific care actions based on the etiology, e.g., purchase an AED or Chest Compression System (LUCAS).

Although described primarily in the context of detection of SCA, system 2 may be used to detection any of a number of acute health events of patient 4. For example, system 2 may be used to detect stroke. Stroke can often present in the form of facial droop. This change in facial tone could be identified using facial image processing on a computing device 12, e.g., a smartphone, or IoT 30. Such image processing could be a primary indicator of possible stroke or a part of a confirmation after another device indications changes related to stroke.

Some computing devices 12, e.g., smartphones, include facial processing for access, e.g., face ID, and are accessed in this manner frequently throughout the day. Processing circuitry, e.g., of the computing device, may analyze the facial images to detect subtle changes in facial tone over time. The processing circuitry could detect possible stroke, and various devices of system 2 could provide alerts as described herein.

In some examples, in response to detection based on the camera images, the device could output a series of prompts (audible and/or visual) to access a current state of patient 4. Patient 4 could be prompted to repeat a phrase or answer audible questions to assess cognitive ability. The device could use additional motion processing to further verify the state of patient 4, e.g., using an accelerometer of computing device 12A and/or 12B. Changes in body motion and asymmetry, e.g., of the face and/or body motion, are indictive of stroke. In some examples, the device may ask patient 4 questions. Processing circuitry may analyze the response to detect speech difficulties associated with stroke. In some examples, the alert could include information on where the facial tone has changed, which could aid in diagnosis by guiding care givers 40 to possible primary locations for scans (ex: left side droop=right side clot).

As described herein, processing circuitry of one or more devices of system 2, e.g., IMD 10, edge devices such as computing devices 12 or IoT devices 30, and/or HMS 22 (or other cloud services), may be configured to analyze episode data associated with an acute health event, such a ventricular tachyarrhythmia or SCA, detected by IMD 10. The episode data may include ECG and other physiological parameter data collected by IMD 10 for the event, e.g., leading up to, during, and/or after the event. As described herein, the analysis may include the application of a second set of rules (as opposed to a first set applied by IMD 10), e.g., a machine learning model or other artificial intelligence algorithm, decision trees, and/or thresholds, to the episode data and, in some cases, a variety of patient data collected by devices of system 2.

The initial detection of a ventricular tachyarrhythmia episode by IMD 10 may be based on a first set rules relating to rate and regularity of RR intervals as well as morphological features of the ECG, e.g., of the R-wave. These rules may lead to inappropriate detections due to oversensing R-waves. Further, true ventricular tachyarrhythmia can be of supraventricular origin, e.g., SVT, or ventricular origin such as VF and VT. VT may be monomorphic or polymorphic. In general, polymorphic VT (PVT) and VF are life threatening, while monomorphic VT (MVT) are life threatening if sustained for durations on the order of minutes, and SVTs are generally not life threatening unless sustained for greater than 1 hour. The techniques of this disclosure may include use of a second set of rules that includes machine learning models or other AI algorithms to improve accuracy of classification of these different forms of ventricular tachyarrhythmia that may be detected by IMDs.

Although described herein primarily the context of ventricular tachyarrhythmias and SCA, the techniques of this disclosure relating to application of a second set of rules to episode data to classify an episode detected by a medical device may be applicable to any health event detectable by a medical device, including heart failure, myocardial infarction, stroke, seizure, falls, and glucose excursions. Further, although described primarily in the context of examples in which a computing device, such as computing device 12A, applies the second set of rules to the episode data, in some examples other devices may apply the second set of rules, such as IMD 10, computing device 12B, IoT devices 30, or HMS 22. In general, the techniques may be applied in any situation in which feedback from the application of a more processing resource intense second set of rules to episode data may be used to dynamically provide patient-specific adjustments of sensitivity and/or specificity of a first set of rules employed by a medical device to detect health events.

The sensitivity and/or specificity of IMD 10, e.g., an ICM, for detecting life threatening ventricular tachyarrhythmias, for example, is patient dependent, e.g., varies with ECG signal amplitude/characteristics, and a single first set of rules may not provide the best performance for all patients-resulting in either too many false transmissions (e.g., oversensing, noise, SVT which are not life threatening) or delayed/missed identification of true life threatening ventricular arrhythmias. As discussed above, in some examples, processing circuitry of system 2 may select, set, or modify the first and/or second sets of rules for a particular patient based on patient etiology, e.g., prior ventricular tachyarrhythmia, prior myocardial infarction, prior stroke, long QT syndrome, or presence of heart failure. Processing circuitry may receive etiology information as user input or by accessing EHR 24. In some examples, user input of etiology information may include selection of an indication, e.g., reason for implanting/monitoring with, IMD 10 during programming of IMD 10. The indication may be VT/VF or increased risk of VT/VF, in which case the processing circuitry may configure the first and/or second sets of rules to have relatively high sensitivity to VT/VF. In some examples, processing circuitry implementing HMS 22 may configure the rules for the patient, e.g., by communication with IMD 10 and/or computing device 12 via network 16, based on etiology information, e.g., received from clinician computing device(s) 38 via network 16.

Patient-specific, dynamic updates of the sensitivity/specificity of the first set of rules may improve the ability of system 2 to detect health events, and limit unnecessary alerts, communication, application of the second set of rules, and other actions of system 2 in response to detection of a health event by a medical devices, as described herein. Furthermore, in some examples, real-time communication between a medical device and a device applying the second set of rules may allow the second device to co-analyze episode data such that unnecessary data relating to non-life threatening episodes is not stored by the medical device. A classification of an episode for which transmission of the episode data from the medical device to the computing device was unnecessary may comprise a classification of the episode as at least one of non-life threatening, not requiring a medical response, or false, as examples.

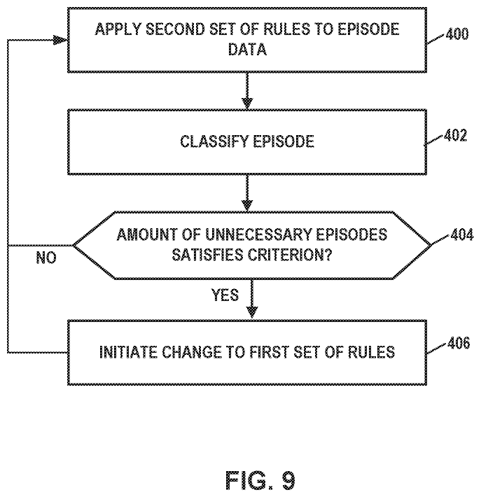

FIG. 9 is a flow diagram illustrating an example operation for initiating a change to rules used by a medical device to identify episodes. The example operation of FIG. 9 is described as being performed by computing device 12A and with respect to ventricular tachyarrhythmia episodes, but could be performed by any one or more devices of system 2 and for any type of health event detectable by an implantable or wearable medical device, as indicated above.

According to the example operation of FIG. 9, processing circuitry 130 of computing device 12A, e.g., rules engine 172, applies a second set of rules 196 to episode data received from IMD 10 relating to an episode (400). As described herein, episode data is stored by IMD 10 for episodes in response to detecting a health event, e.g., a ventricular tachyarrhythmia, based on application of a first set of rules 84 by a rules engine 74 of IMD 10 to sensed data 82 sensed by IMD 10.

Processing circuitry 130 of computing device 12 may classify the episode as one of a plurality of classifications based on the application of the second set of rules to the episode data (402). At least one of the plurality of classifications comprises a classification for which transmission of the episode data from the medical device to the computing device was unnecessary. For example, it may be unnecessary for IMD to record and transmit episode data for ventricular tachyarrhythmia episodes that would be classified by the second set of rules as SVT, noise, or oversensing.

Processing circuitry 130 may also determine whether an amount of unnecessary episodes satisfies at least one criterion, such as a number of consecutive unnecessary episodes, or a number unnecessary episodes within a predetermined number of most recent episodes (404). If the amount of unnecessary episodes does not satisfy the criterion (N of 404), processing circuitry 130 may continue to apply the second set of rules to episode data received from IMD 10 (400). If the amount of unnecessary episodes does satisfy the criterion (Y of 404), processing circuitry 130 may initiate a change to the first set of rules 84 applied by IMD 10 (406).

Initiating the change to the first set of rules may include transmitting an instruction to IMD 10 and/or change request to HMS 22 or another cloud-based service. In the latter case, the change may be automatically implemented by the cloud-based service, or proposed to a clinician or other user. The instruction to IMD 10 may comprise a changed value for at least one of the first set of rules or an instruction to change to a different version of the first set of rules. For example, computing device 12 may instruct IMD 10 to change between different versions of the first set of rules having relatively higher or lower sensitivity based on whether the number of unnecessary episodes satisfies the criterion. In some examples, IMD 10 may use a relatively higher sensitivity version of the first set of rules until instructed by change by computing device 12. In some examples, IMD 10 may revert to a higher sensitivity set of rules after operating at a lower sensitivity for a predetermined amount of time, or if the second set of rules classifies a number of true episodes at the lower sensitivity setting.

In some examples, computing device 12 may change the rules used by IMD 10 to balance risk of false positives while maintaining high sensitivity for true VT/VF rhythms. The computing device may “learn” the best settings from data it receives from IMD 10 and update the rules used by IMD 10 over time. A simple solution could be switching the first set of rules from a pre-determined set of “Ultra-high sensitivity” parameter settings to another pre-determined set of “High sensitivity” parameter settings based on computing device 12 recognizing a certain rate of false positive ventricular tachyarrhythmia detections from data transmitted from IMD 10. Changes to the first set of rules may include as examples, changes to at least one of a number of cardiac intervals to detect threshold, a heart rate threshold, a criterion for detecting R-waves, such as a sensitivity threshold or an auto-adjusting threshold parameter, or a noise rejection setting, such as a noise rejection setting for a VF counter or an onset counter which starts a sustained duration. Changes to the first set of rules may include as further examples, other sensing and detection parameters, such as blanking interval, sensing threshold decay delay (increase in case of T-wave oversensing), VT detection interval, fast VT (FVT) detection interval, VT number of intervals to detect, FVT number of intervals to detect, etc. In some examples, the initial set of rules, a criterion for changing the set of rules, and any changes to the set of rules, may be user programmed. In some examples, the initial set of rules may be selected for a patient based on clinical history, indication for use of IMD 10, or other factors.

In some examples, the computing device or other device implementing the second set of rules may receive feedback from a user, e.g., patient 4, a caregiver, family member, or responder, that indicates that the patient is “OK” despite an episode being classified as life-threatening or requiring some intervention. In some examples, the device may use the episode data for such episodes to modify the second set of rules, e.g., self-learning by an AI algorithm. In some examples, the device may use the episode data for such episodes to modify the second set of rules based on an amount of such false positive episodes satisfying one or more criteria.

To detect acute health events, such as SCA or other potentially life-threatening arrhythmias, a set of rules (e.g., a first set of rules implemented by IMD 10 or another sensor device) must effectively balance sensitivity (i.e., not missing the presence of a true event) against specificity (i.e., appropriately ruling out false positive events). Programmable settings control the rules and attempt to strike a sensitivity/specificity balance that is appropriate for the patient. However, there may be clinical circumstances, e.g., observable by the sensor device or other processing circuitry of system 2, that may alter what is an appropriate balance for the patient.

For example, processing circuitry of system 2 (e.g., processing circuitry 50 of IMD 10, processing circuitry 130 of computing device 12, or processing circuitry of computing system 20 implementing HMS 22) may determine that a current state or condition of the patient is one associated with increased or decreased risk of life-threatening arrhythmia or another health event, and adjust a set of rules to rebalance sensitivity and specificity based on that determination. If risk of the target health event is perceived to be increased, the processing circuitry of system 2 may modify a set of rules, e.g., a first set of rules implemented by IMD 10, to increase sensitivity to the target health event. The modification of the set of rules to increase sensitivity will typically result in decreased specificity, which may be acceptable due to the perceived increased risk of the target health event. If risk of the target health event is perceived to be decreased, the processing circuitry of system 2 may modify the set of rules, e.g., the first set of rules implemented by IMD 10, to increase specificity. The modification of the set of rules to increase specificity will typically result in decreased sensitivity, which may be acceptable due to the perceived decreased risk of the target health event. In the case of a set of rules detect potentially lethal tachyarrhythmias, modifications to rebalance sensitivity/specificity may include modifying a number of intervals to detect (NID), including modifying the number of beats and/or the rate, changing a machine learned model and/or a threshold probability applied to an output of a machine learned model. In some examples, the processing circuitry may evaluate the current state/condition patient for changing sensitivity/specificity at a first periodical interval, but will wait a second, longer period after changing before reevaluating the patient.

The processing circuitry may determine the current state/condition of the patient based on parameters sensed by IMD 10, e.g., an ICM, a wearable device, or any other device described herein, or input received from the patient (e.g., via a computing device 12) or any other user. The parameters that may indicate increased/decreased risk of SCA include T-wave alternans, QT prolongation, T-wave inversion, QRS axis shift, ST elevation or depression, presence or number of premature ventricular contractions (PVCs), presence or number of non-sustained tachyarrhythmias, or other parameters derived from an ECG or other cardiac signal, e.g., sensed by IMD 10. The parameters that may indicate increased risk of SCA include indicators of ischemia, such as heart sounds (e.g., change in amplitude or onset of a new heart sounds such as S3 or S4), biochemical parameters (e.g., change in any such as glucose, creatinine, or potassium), or presence or number of PVCs and/or NSTs. The parameters that may indicate increased risk of SCA include parameters indicative of heart failure status/progression, such as respiration, fluid congestion, blood pressure, blood oxygen saturation, patient activity (motion) and/or patient posture.

For example, if a patient is at risk of a heart failure decompensation event, e.g., as determined by HMS 22 implementing a technique for monitoring heart failure using multiple parameters determined from signals sensed by IMD 10, the patient is also more likely to experience a life-threatening arrhythmia. In this scenario, rules used by IMD 10 to detect life-threatening arrhythmias may be more appropriately shifted to be highly sensitive (to avoid missing a potential arrhythmia) even if it comes at the expense of specificity. Techniques for monitoring heart failure of patients include the TRIAGE-HF™ tool available from Medtronic, Inc., as well as those described in U.S. Publication No. 2012/0253207, by Sarkar et al. and Cowie et al., “Development and validation of an integrated diagnostic algorithm derived from parameters monitored in implantable devices for identifying patients at risk for heart failure hospitalization in an ambulatory setting,” European Heart Journal (2013) 34, 2472-2480, which are incorporated herein by reference.

In some examples, a medical device that senses patient parameters to detect health events, such as IMD 10, may implement a screening algorithm to determine whether to store and/or transmit episode data for detected health events. Such screening algorithms may affect the sensitivity and specificity of health event detection by the medical device, as well as the longevity of the medical device, e.g., because waking up the device to communicate and, more generally, processing data, consume power of the medical device.

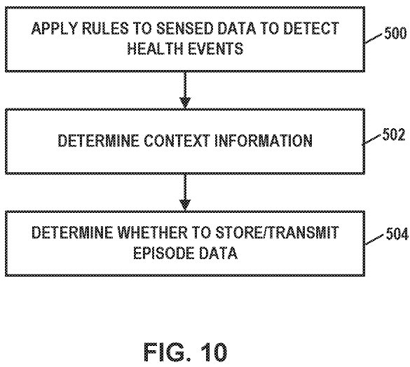

FIG. 10 is a flow diagram illustrating an example operation of a medical device for determining whether to store and/or transmit episode data. Although described as being performed by IMD 10, the example operation of FIG. 10 may be performed by any implantable or wearable medical device described herein.

According to the example operation of FIG. 10, processing circuitry 50 of IMD 10 applies rules 84 to sensed data 82 to detect health events (500). Processing 50 further determines, for each of the health events, context information for the application of the set of rules to data sensed by the medical device (502). Processing circuitry 50 determines whether to store episode data for the health event in memory 52 and/or whether to transmit the episode data, e.g., to computing device 12 or another device of system 2 as described herein, based on the context information (504).

Time of day is one example of context information. Certain health events may be more or less likely at certain times of day, e.g., ventricular tachyarrhythmias may be more likely to occur in morning with sympathetic surge while atrial arrhythmias are vagally mediated and may be more likely in evening. Another example of context information is density of health event detections, e.g., frequency, such as number per day or month. False positive health event detections often come in clusters, while true health events are more isolated in time. Another example of context information is patient location, as location may be correlated with certain patient activities or exposures to other sources of noise in the ECG or other signals used to detect health events. Similarly, patient posture and activity level may be examples of context information due to their potential to cause noise or artifacts in sensor signals or to detect falls in the case of true hemodynamically comprised ventricular tachyarrhythmias. Other examples of context information is the presence or burden of AF, unusual durations for health events of a given type, classification of one or more previously detected health events by another device using another set of rules, regularity of R-R intervals during the event, rate of R-R intervals during the event, or electrocardiogram morphology. Another example of context information is status information of patient 4 input by patient 4 or another user, such as a caregiver, family member, or responder. The input could be via computing devices 12A or 12B, or an IoT device 30, or via tapping on IMD 10. The input could be by voice, e.g., recognition of speech of the patient or other user. In some examples, processing circuitry 50 determines whether to store and/or transmit episode data based on a weighted combination of two or more of these types of context information.

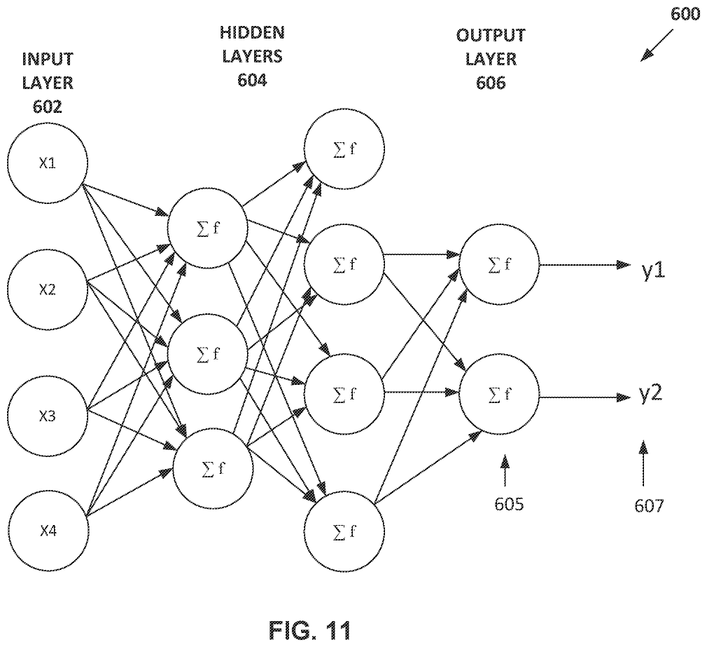

FIG. 11 is a conceptual diagram illustrating an example machine learning model 600 configured to determine an extent to which patient parameter data is indicative of an acute health event, such as SCA. Machine learning model 600 is an example of a set of rules, e.g., a second set of rules implemented by rules engine 172 of computing device 12 in wireless communication with IMD 10, as discussed above. Machine learning model 600 is an example of a deep learning model, or deep learning algorithm, trained to determine whether a particular set of patient parameter data indicates the presence of an acute health event, e.g., whether a particular segment of ECG signal data indicates SCA. One or more of IMD 10, external device 12, an IoT device 30, or a computing system 20 may train, store, and/or utilize machine learning model 600, but other devices may apply inputs associated with a particular patient to machine learning model 600 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 11, machine learning model 600 may include three layers. These three layers include input layer 602, hidden layer 604, and output layer 606. Output layer 606 comprises the output from the transfer function 605 of output layer 606. Input layer 602 represents each of the input values X1 through X4 provided to machine learning model 1500. The number of inputs may be less than or greater than 4, including much greater than 4, e.g., hundreds or thousands. In some examples, the input values may any of the of values input into a machine learning model, as described above. In some examples, input values may include samples of an ECG signal. In addition, in some examples input values of machine learning model 600 may include additional data, such as data relating to one or more additional parameters of patient 4.

Each of the input values for each node in the input layer 602 is provided to each node of hidden layer 604. In the example of FIG. 11, hidden layers 604 include two layers, one layer having four nodes and the other layer having three nodes, but fewer or greater number of nodes may be used in other examples. Each input from input layer 602 is multiplied by a weight and then summed at each node of hidden layers 604. During training of machine learning model 600, the weights for each input are adjusted to establish the relationship between the inputs, e.g., input ECG segment, to determining whether a particular set of inputs represents an acute health event and/or determining a score indicative of whether a set of inputs may be representative of SCA or another acute health event. In some examples, one hidden layer may be incorporated into machine learning model 600, or three or more hidden layers may be incorporated into machine learning model 600, where each layer includes the same or different number of nodes.

The result of each node within hidden layers 604 is applied to the transfer function of output layer 606. The transfer function may be liner or non-linear, depending on the number of layers within machine learning model 600. Example non-linear transfer functions may be a sigmoid function or a rectifier function. The output 607 of the transfer function may be a classification that indicates whether the particular ECG segment or other input set represents an acute health event and/or a score indicative of an extent to which the input data set represents an acute health event.

By applying the ECG signal data and/or other patient parameter data to a machine learning model, such as machine learning model 600, processing circuitry, such as processing circuitry 130 of computing device 12, is able to determine a patient is experiencing or will soon experience an acute health event with great accuracy, specificity, and sensitivity. This may facilitate determinations of risk of sudden cardiac death, and may lead to alerts and other interventions as described herein. Machine learning model 600 may correspond to any one or more of rules 84, rules 196, and rules 250 described herein.

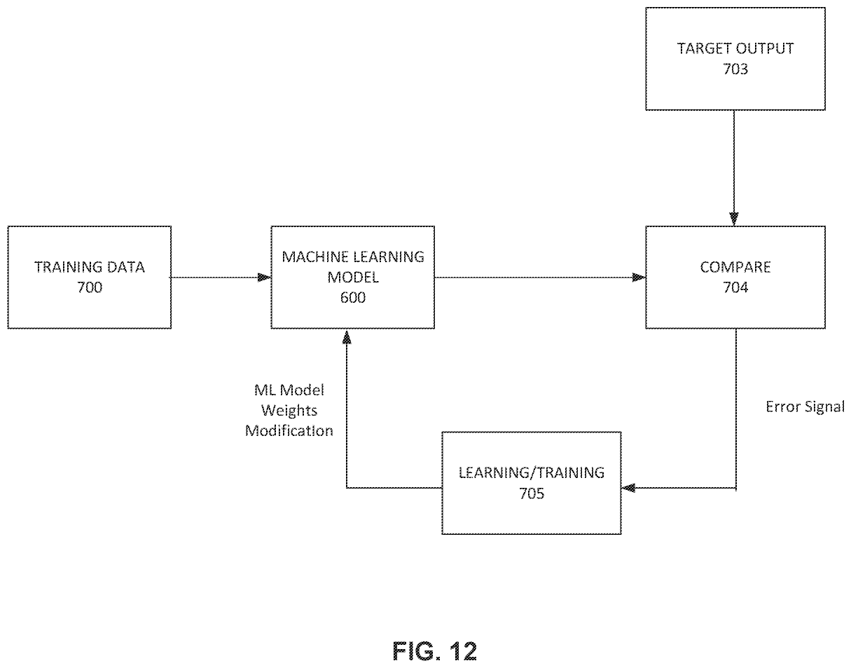

FIG. 12 is an example of a machine learning model 600 being trained using supervised and/or reinforcement learning techniques. Machine learning model 600 may be implemented using any number of models for supervised and/or reinforcement learning, such as but not limited to, an artificial neural network, a decision tree, naïve Bayes network, support vector machine, or k-nearest neighbor model, to name only a few of the examples discussed above. In some examples, processing circuitry one or more of IMD 10, external device 12, an IoT device, and/or computing system(s) 20 (e.g., rules configuration modules 174 and/or 234) initially trains the machine learning model 600 based on training set data 700 including numerous instances of input data corresponding to acute health events and non-acute health events, e.g., as labeled by an expert. A prediction or classification by the machine learning model 600 may be compared 704 to the target output 703, e.g., as determined based on the label. Based on an error signal representing the comparison, the processing circuitry implementing a learning/training function 705 may send or apply a modification to weights of machine learning model 600 or otherwise modify/update the machine learning model 600. For example, one or more of IMD 10, external device 12, IoT device 30, and/or computing system(s) 20 may, for each training instance in the training set 700, modify machine learning model 600 to change a score generated by the machine learning model 600 in response to data applied to the machine learning model 600.

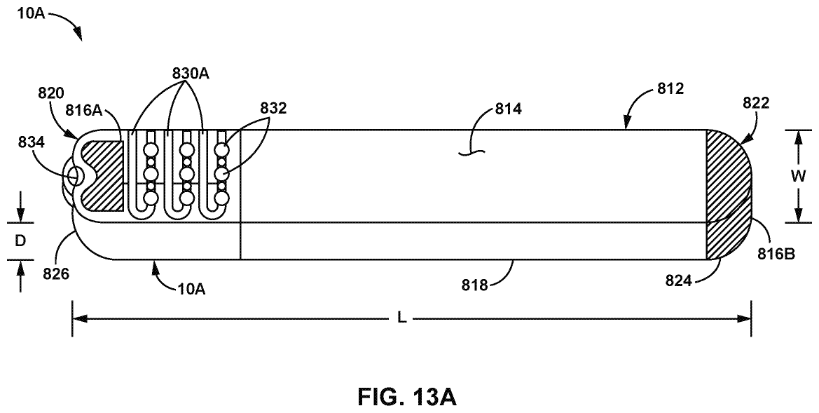

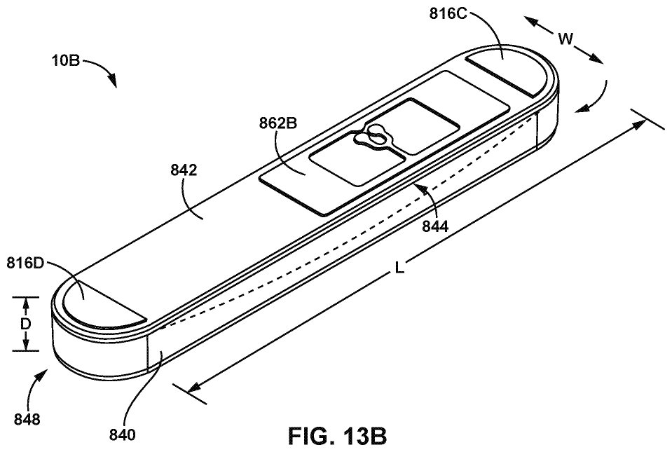

FIG. 13A is a perspective drawing illustrating an IMD 10A, which may be an example configuration of IMD 10 of FIGS. 1 and 2 as an ICM. In the example shown in FIG. 13A, IMD 10A may be embodied as a monitoring device having housing 812, proximal electrode 816A and distal electrode 816B. Housing 812 may further comprise first major surface 814, second major surface 818, proximal end 820, and distal end 822. Housing 812 encloses electronic circuitry located inside the IMD 10A and protects the circuitry contained therein from body fluids. Housing 812 may be hermetically sealed and configured for subcutaneous implantation. Electrical feedthroughs provide electrical connection of electrodes 816A and 816B.

In the example shown in FIG. 13A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 13A includes radial asymmetries (notably, the rectangular shape) along the longitudinal axis that maintains the device in the proper orientation following insertion. For example, the spacing between proximal electrode 816A and distal electrode 816B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length/may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 13A, once inserted within the patient, the first major surface 814 faces outward, toward the skin of the patient while the second major surface 818 is located opposite the first major surface 814. In addition, in the example shown in FIG. 13A, proximal end 820 and distal end 822 are rounded to reduce discomfort and irritation to surrounding tissue once inserted under the skin of the patient. IMD 10A, including instrument and method for inserting IMD 10 is described, for example, in U.S. Patent Publication No. 2014/0276928, incorporated herein by reference in its entirety.

Proximal electrode 816A is at or proximate to proximal end 820, and distal electrode 816B is at or proximate to distal end 822. Proximal electrode 816A and distal electrode 816B are used to sense cardiac EGM signals, e.g., ECG signals, thoracically outside the ribcage, which may be sub-muscularly or subcutaneously. Cardiac signals may be stored in a memory of IMD 10A, and data may be transmitted via integrated antenna 830A to another device, which may be another implantable device or an external device, such as external device 812. In some example, electrodes 816A and 816B may additionally or alternatively be used for sensing any bio-potential signal of interest, which may be, for example, an EGM, EEG, EMG, or a nerve signal, or for measuring impedance, from any implanted location.

In the example shown in FIG. 13A, proximal electrode 816A is at or in close proximity to the proximal end 820 and distal electrode 816B is at or in close proximity to distal end 822. In this example, distal electrode 816B is not limited to a flattened, outward facing surface, but may extend from first major surface 814 around rounded edges 824 and/or end surface 826 and onto the second major surface 818 so that the electrode 816B has a three-dimensional curved configuration. In some examples, electrode 816B is an uninsulated portion of a metallic, e.g., titanium, part of housing 812.

In the example shown in FIG. 13A, proximal electrode 816A is located on first major surface 814 and is substantially flat, and outward facing. However, in other examples proximal electrode 816A may utilize the three dimensional curved configuration of distal electrode 816B, providing a three dimensional proximal electrode (not shown in this example). Similarly, in other examples distal electrode 816B may utilize a substantially flat, outward facing electrode located on first major surface 814 similar to that shown with respect to proximal electrode 816A.

The various electrode configurations allow for configurations in which proximal electrode 816A and distal electrode 816B are located on both first major surface 814 and second major surface 818. In other configurations, such as that shown in FIG. 13A, only one of proximal electrode 816A and distal electrode 816B is located on both major surfaces 814 and 818, and in still other configurations both proximal electrode 816A and distal electrode 816B are located on one of the first major surface 814 or the second major surface 818 (e.g., proximal electrode 816A located on first major surface 814 while distal electrode 816B is located on second major surface 818). In another example, IMD 10A may include electrodes on both major surface 814 and 818 at or near the proximal and distal ends of the device, such that a total of four electrodes are included on IMD 10A. Electrodes 816A and 816B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 13A, proximal end 820 includes a header assembly 828 that includes one or more of proximal electrode 816A, integrated antenna 830A, anti-migration projections 382, and/or suture hole 834. Integrated antenna 830A is located on the same major surface (i.e., first major surface 814) as proximal electrode 816A and is also included as part of header assembly 828. Integrated antenna 830A allows IMD 10A to transmit and/or receive data. In other examples, integrated antenna 830A may be formed on the opposite major surface as proximal electrode 816A, or may be incorporated within the housing 812 of IMD 10A. In the example shown in FIG. 13A, anti-migration projections 832 are located adjacent to integrated antenna 830A and protrude away from first major surface 814 to prevent longitudinal movement of the device. In the example shown in FIG. 13A, anti-migration projections 832 include a plurality (e.g., nine) small bumps or protrusions extending away from first major surface 814. As discussed above, in other examples anti-migration projections 832 may be located on the opposite major surface as proximal electrode 816A and/or integrated antenna 830A. In addition, in the example shown in FIG. 13A, header assembly 828 includes suture hole 834, which provides another means of securing IMD 10A to the patient to prevent movement following insertion. In the example shown, suture hole 834 is located adjacent to proximal electrode 816A. In one example, header assembly 828 is a molded header assembly made from a polymeric or plastic material, which may be integrated or separable from the main portion of IMD 10A.

FIG. 13B is a perspective drawing illustrating another IMD 10B, which may be another example configuration of IMD 10 from FIGS. 1 and 2 as an ICM. IMD 10B of FIG. 13B may be configured substantially similarly to IMD 10A of FIG. 13A, with differences between them discussed herein.

IMD 10B may include a leadless, subcutaneously-implantable monitoring device, e.g. an ICM. IMD 10B includes housing having a base 840 and an insulative cover 842. Proximal electrode 816C and distal electrode 816D may be formed or placed on an outer surface of cover 842. Various circuitries and components of IMD 10B, e.g., described above with respect to FIG. 2, may be formed or placed on an inner surface of cover 842, or within base 840. In some examples, a battery or other power source of IMD 10B may be included within base 840. In the illustrated example, antenna 830B is formed or placed on the outer surface of cover 842, but may be formed or placed on the inner surface in some examples. In some examples, insulative cover 842 may be positioned over an open base 840 such that base 840 and cover 842 enclose the circuitries and other components and protect them from fluids such as body fluids. The housing including base 840 and insulative cover 842 may be hermetically sealed and configured for subcutaneous implantation.

Circuitries and components may be formed on the inner side of insulative cover 842, such as by using flip-chip technology. Insulative cover 842 may be flipped onto a base 840. When flipped and placed onto base 840, the components of IMD 10B formed on the inner side of insulative cover 842 may be positioned in a gap 844 defined by base 840. Electrodes 816C and 816D and antenna 830B may be electrically connected to circuitry formed on the inner side of insulative cover 842 through one or more vias (not shown) formed through insulative cover 842. Insulative cover 842 may be formed of sapphire (i.e., corundum), glass, parylene, and/or any other suitable insulating material. Base 840 may be formed from titanium or any other suitable material (e.g., a biocompatible material). Electrodes 816C and 816D may be formed from any of stainless steel, titanium, platinum, iridium, or alloys thereof. In addition, electrodes 816C and 816D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 13A. For example, the spacing between proximal electrode 816C and distal electrode 816D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 13B, once inserted subcutaneously within the patient, outer surface of cover 842 faces outward, toward the skin of the patient. In addition, as shown in FIG. 13B, proximal end 846 and distal end 848 are rounded to reduce discomfort and irritation to surrounding tissue once inserted under the skin of the patient. In addition, edges of IMD 10B may be rounded.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1. A method comprising: applying, by processing circuitry of a computing device configured to communicate with a medical device, a second set of rules to episode data received from the medical device for each of a plurality of episodes, the episode data stored by the medical device for the episodes in response to detecting a health event based on application of a first set of rules by the medical device to sensed data sensed by the medical device; classifying, by the processing circuitry, each episode of the plurality of episodes as one of a plurality of classifications based on the application of the second set of rules to the episode data, wherein at least one of the plurality of classifications comprises a classification for which transmission of the episode data from the medical device to the computing device was unnecessary; determining, by the processing circuitry, that an amount of the plurality of episodes classified as the classification for which transmission of the episode data from the medical device to the computing device was unnecessary satisfies at least one criterion; and initiating, by the processing circuitry, a change to the first set of rules applied by the medical device based on the determination.

Example 2. The method of example 1, wherein the medical device comprises an implantable medical device.

Example 3. The method of example 2, wherein the implantable medical device comprises an insertable cardiac monitor.

Example 4. The method of any one or more of examples 1-3, wherein the second set of rules comprises an artificial intelligence algorithm.

Example 5. The method of any one or more of examples 1-4, wherein the plurality of episodes comprises a plurality of ventricular tachyarrhythmia episodes.

Example 6. The method of example 5, wherein the classification for which transmission of the episode data from the medical device to the computing device was unnecessary comprises at least one of noise, oversensing, or supraventricular tachycardia.

Example 7. The method of any one or more of examples 1-6, wherein initiating the change to the first set of rules comprises transmitting an instruction from the computing device to the medical device.

Example 8. The method of example 7, wherein the instruction comprises a changed value for at least one rule of the first set of rules.

Example 9. The method of example 7, wherein the instruction comprises an instruction to select a different version of the first set of rules.

Example 10. The method of any one or more of examples 1-6, wherein

initiating the change to the first set of rules comprises transmitting a request to change the first set of rules from the computing device to a cloud-based health monitoring system.

Example 11. The method of any one or more of examples 1-10, wherein initiating the change to the first set of rules comprises initiating a change to at least one of a number of cardiac intervals threshold, a heart rate threshold, a criterion for detecting R-waves, or a noise rejection setting.

Example 12. The method of any one or more of examples 1-11, wherein the classification for which transmission of the episode data from the medical device to the computing device was unnecessary comprises a classification of the episode as at least one of non-life threatening, not requiring a medical response, or a false positive.

Example 13. A method comprising: applying, by processing circuitry of at least one of a smartphone or a smartwatch configured to wirelessly communicate with an insertable cardiac monitor, a second set of rules to episode data received from the medical device for each of a plurality of episodes, the episode data stored by the medical device for the episodes in response to detecting a ventricular tachyarrhythmia based on application of a first set of rules by the medical device to sensed data sensed by the medical device, wherein the second set of rules comprises an artificial intelligence algorithm; classifying, by the processing circuitry, each episode of the plurality of episodes as one of a plurality of classifications based on the application of the second set of rules to the episode data, wherein at least one of the plurality of classifications comprises a classification for which transmission of the episode data from the medical device to the at least one of the smartphone or the smartwatch was unnecessary, the classification for which transmission of the episode data from the medical device to the at least one of the smartphone or the smartwatch was unnecessary comprises at least one of noise, oversensing, or supraventricular tachycardia; determining, by the processing circuitry, that an amount of the plurality of episodes classified as the classification for which transmission of the episode data from the medical device to the at least one of the smartphone or the smartwatch was unnecessary satisfies at least one criterion; and initiating, by the processing circuitry, a change to the first set of rules applied by the medical device based on the determination.

Example 14. A computing device comprising: processing circuitry; and memory comprising program instructions that, when executed by the processing circuitry, cause the processing circuitry to perform the method of any of examples 1-13.

Example 15. The computing device of example 13, wherein the computing device comprises a smartphone, smartwatch, or Internet of Things device.

Example 16. A computer-readable storage medium comprising instructions that, when executed by processing circuitry, cause the processing circuitry to perform the method of any of examples 1-13.

Example 17. A method comprising: applying, by processing circuitry of a medical device, a set of rules to data sensed by the medical device to detect a plurality of health events of a patient; for each of the plurality of health events detected by the medical device, determining, by the processing circuitry, context information for the application of the set of rules to data sensed by the medical device; and determining, by the processing circuitry, whether to at least one of store episode data for the health event in a memory of the medical device or transmit the episode data for the health event to a computing device based on the context information.

Example 18. The method of example 17, wherein the context information comprises one or more of: time of day; density of detected health events; location of the patient; activity of the patient; posture of the patient; presence of atrial fibrillation; classification of one or more previously detected health events by another device using another set of rules; duration of previously detected health events; regularity of R-R intervals; rate of R-R intervals; electrocardiogram morphology; or user-inputted patient status.

Example 19. The method of example 18, wherein determining whether to at least one of store episode data or transmit the episode data based on the context information comprises determining whether to at least one of store episode data or transmit the episode data based on a weighted combination of two or more of: the time of day; the density of detected health events; the location of the patient; the activity of the patient; the posture of the patient; the presence of atrial fibrillation; the classification of one or more previously detected health events by another device using another set of rules; the duration of previously detected health events; the regularity of R-R intervals; the rate of R-R intervals; the electrocardiogram morphology; or user-inputted patient status.

Example 20. A medical device comprising: processing circuitry; and memory comprising program instructions that, when executed by the processing circuitry, cause the processing circuitry to perform the method of any of examples 16-18.

Example 21. The medical device of example 20, wherein the medical device comprises an implantable medical device.

Example 22. The medical device of example 21, wherein the implantable medical device comprises an insertable cardiac monitor.

Example 23. A computer-readable storage medium comprising instructions that, when executed by processing circuitry, cause the processing circuitry to perform the method of any of examples 17-19.

Example 24. A computing device comprising: communication circuitry; processing circuitry; and memory comprising program instructions that, when executed by the processing circuitry, cause the processing circuitry to: apply a second set of rules to episode data wirelessly received from a medical device for each of a plurality of episodes via the communication circuitry, the episode data stored by the medical device for the episodes in response to detecting a health event based on application of a first set of rules by the medical device to sensed data sensed by the medical device; classify each episode of the plurality of episodes as one of a plurality of classifications based on the application of the second set of rules to the episode data, wherein at least one of the plurality of classifications comprises a classification for which transmission of the episode data from the medical device to the computing device was unnecessary; determine that an amount of the plurality of episodes classified as the classification for which transmission of the episode data from the medical device to the computing device was unnecessary satisfies at least one criterion; and initiate a change to the first set of rules applied by the medical device based on the determination.

Example 25. The computing device of example 1, wherein the second set of rules comprises a machine learning model.

Example 26. The computing device of example 1 or 2, wherein the plurality of episodes comprises a plurality of ventricular tachyarrhythmia episodes.

Example 27. The computing device of example 3, wherein the classification for which transmission of the episode data from the medical device to the computing device was unnecessary comprises at least one of noise, oversensing, or supraventricular tachycardia.

Example 28. The computing device of any one or more of examples 1-4, wherein the processing circuitry is configured to wirelessly transmit an instruction to the medical device via the communication circuitry to initiate the change to the first set of rules.

Example 29. The computing device of example 5, wherein the instruction comprises a changed value for at least one rule of the first set of rules.

Example 30. The computing device of example 5, wherein the instruction comprises an instruction to select a different version of the first set of rules.

Example 31. The computing device of any one or more of examples 1-4, wherein the processing circuitry is configured to transmit a request to change the first set of rules from the computing device to a cloud-based health monitoring system via the communication circuitry to initiate the change to the first set of rules.

Example 32. The computing device of any one or more of examples 1-8, wherein to initiate the change to the first set of rules, the processing circuitry is configured to initiate a change to at least one of a number of cardiac intervals threshold, a heart rate threshold, a criterion for detecting R-waves, or a noise rejection setting.

Example 33. The computing device of any one or more of examples 1-9, wherein the classification for which transmission of the episode data from the medical device to the computing device was unnecessary comprises a classification of the episode as at least one of non-life threatening, not requiring a medical response, or a false positive.

Example 34. A system comprising: the computing device of any one or more of examples 1-10; and the medical device.

Example 35. The system of example 11, wherein the medical device comprises an implantable medical device.

Example 36. The system of example 12, wherein the implantable medical device comprises an insertable cardiac monitor.

Example 37. The system of example 13, wherein the processing circuitry and memory of the computing device comprises first processing circuitry and first memory, and the insertable cardiac monitor comprises: a hermetically sealed housing configured for subcutaneous implantation within the patient, wherein the housing has a length from a first end to a second end, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm; second processing circuitry within the housing; a power source within the housing and operatively coupled to the processing circuitry; a second memory within the housing and operatively coupled to the processing circuitry; sensing circuitry within the housing and operatively coupled to the processing circuitry; a first electrode at or proximate the first end of the housing and operatively coupled to the sensing circuitry; and a second electrode at or proximate the second end of the housing and operatively coupled to the sensing circuitry.

Example 38. The system of any one or more of examples 11-14, wherein the computing device comprises at least one of a smartphone, a smartwatch, or an Internet of Things device.

Example 39. A system comprising: a medical device configured to sense parameter data of a patient, including at least an electrocardiogram of the patient; and processing circuitry configured to: detect an acute health event of the patient based on application of a set of rules to a first set of the parameter data; determine that a risk of the acute health event is changed based on a second set of the parameter data; and adjust at least one of a sensitivity or specificity of the set of rules based on the determination that the risk of the acute health event is changed.

Example 40. The system of example 37, wherein the processing circuitry comprises processing circuitry of at least one of the medical device, a computing device configured to wirelessly communicate with the medical device, or a cloud-based computing system configured to communicate with the medical device.

Example 41. The system of example 37 or 38, wherein the medical device comprises an insertable cardiac monitor.

Example 42. The system of any one or more of examples 37-39, wherein the acute health event comprises cardiac arrhythmia, and to determine that the risk of the acute health has changed the processing circuitry is configured to determine that a heart failure status of the patient has changed.

Various examples have been described. These and other examples are within the scope of the following claims.

1. A computing device comprising:
communication circuitry;
processing circuitry; and
memory comprising program instructions that, when executed by the processing circuitry, cause the processing circuitry to:
apply a second set of rules to episode data wirelessly received from a medical device for each of a plurality of episodes via the communication circuitry, the episode data stored by the medical device for the episodes in response to detecting a health event based on application of a first set of rules by the medical device to sensed data sensed by the medical device;
classify each episode of the plurality of episodes as one of a plurality of classifications based on the application of the second set of rules to the episode data, wherein at least one of the plurality of classifications comprises a classification for which transmission of the episode data from the medical device to the computing device was unnecessary, wherein the classification of unnecessary corresponds to at least one of non-life threatening, not requiring a medical response, or a false positive;
determine that an amount of the plurality of episodes classified as the classification for which transmission of the episode data from the medical device to the computing device was unnecessary satisfies at least one criterion; and
transmit a communication via the communication circuitry to initiate a change to the first set of rules applied by the medical device based on the determination.

2. The computing device of claim 1, wherein the second set of rules comprises a machine learning model.

3. The computing device of claim 1, wherein the plurality of episodes comprises a plurality of ventricular tachyarrhythmia episodes.

4. The computing device of claim 3, wherein the classification for which transmission of the episode data from the medical device to the computing device was unnecessary comprises at least one of noise, oversensing, or supraventricular tachycardia.

5. The computing device of claim 1, wherein the transmitted communication comprises an instruction to the medical device to initiate the change to the first set of rules.

6. The computing device of claim 5, wherein the instruction comprises a changed value for at least one rule of the first set of rules.

7. The computing device of claim 5, wherein the instruction comprises an instruction to select a different version of the first set of rules.

8. The computing device of claim 1, wherein the transmitted communication comprises a request to change the first set of rules from the computing device to a cloud-based health monitoring system to initiate the change to the first set of rules.

9. The computing device of claim 1, wherein to initiate the change to the first set of rules, the processing circuitry is configured to initiate a change to at least one of a number of cardiac intervals threshold, a heart rate threshold, a criterion for detecting R-waves, or a noise rejection setting.

10-20. (canceled)

21. A method of controlling the operation of a computing device configured to communicate with a medical device configured to store episode data for episodes in response to detecting a health event of a patient, the method comprising:
applying, by processing circuitry of the computing device, a second set of rules to episode data received from the medical device for each of a plurality of episodes, the episode data stored by the medical device for the episodes in response to detecting a health event based on application of a first set of rules by the medical device to sensed data sensed by the medical device;
classifying, by the processing circuitry, each episode of the plurality of episodes as one of a plurality of classifications based on the application of the second set of rules to the episode data, wherein at least one of the plurality of classifications comprises a classification for which transmission of the episode data from the medical device to the computing device was unnecessary, wherein the classification of unnecessary corresponds to at least one of non-life threatening, not requiring a medical response, or a false positive;
determining, by the processing circuitry, that an amount of the plurality of episodes classified as the classification for which transmission of the episode data from the medical device to the computing device was unnecessary satisfies at least one criterion; and
transmitting, by the processing circuitry and via communication circuitry of the computing device, a communication to initiate a change to the first set of rules applied by the medical device based on the determination.

22. The method of claim 21, wherein the medical device comprises an implantable medical device.

23. The method of claim 22, wherein the implantable medical device comprises an insertable cardiac monitor.

24. The method of claim 21, wherein the second set of rules comprises an artificial intelligence algorithm.

25. The method of claim 21, wherein the plurality of episodes comprises a plurality of ventricular tachyarrhythmia episodes.

26. The method of claim 25, wherein the classification for which transmission of the episode data from the medical device to the computing device was unnecessary comprises at least one of noise, oversensing, or supraventricular tachycardia.

27. The method of claim 21, wherein initiating the change to the first set of rules comprises transmitting, by the processing circuitry and via the communication circuitry of the computing device, an instruction from the computing device to the medical device.

28. The method of claim 27, wherein the instruction comprises a changed value for at least one rule of the first set of rules.

29. The method of claim 27, wherein the instruction comprises an instruction to select a different version of the first set of rules.

30. A system comprising:
a medical device configured to sense parameter data of a patient, including at least an electrocardiogram of the patient; and
processing circuitry configured to:
detect an acute health event of the patient based on application of a set of rules to a first set of the parameter data;
determine that a risk of the acute health event is changed based on a second set of the parameter data; and
adjust at least one of a sensitivity or specificity of the set of rules based on the determination that the risk of the acute health event is changed.

31. A non-transitory computer-readable medium storing instructions that when executed cause processing circuitry to:
apply a second set of rules to episode data wirelessly received from a medical device for each of a plurality of episodes via the communication circuitry, the episode data stored by the medical device for the episodes in response to detecting a health event based on application of a first set of rules by the medical device to sensed data sensed by the medical device;
classify each episode of the plurality of episodes as one of a plurality of classifications based on the application of the second set of rules to the episode data, wherein at least one of the plurality of classifications comprises a classification for which transmission of the episode data from the medical device to the computing device was unnecessary, wherein the classification of unnecessary corresponds to at least one of non-life threatening, not requiring a medical response, or a false positive;
determine that an amount of the plurality of episodes classified as the classification for which transmission of the episode data from the medical device to the computing device was unnecessary satisfies at least one criterion; and
transmit a communication via the communication circuitry to initiate a change to the first set of rules applied by the medical device based on the determination.
